(19) United States
(12) Patent Application Publication
(10) Pub. No.: US 2018/0330817 A1
(43) Pub. Date: Nov. 15, 2018

(54) TRAINING A PATIENT IN MOVING AND WALKING

(71) Applicant: MOTORIKA LIMITED, Hamilton (BM)

(72) Inventors: Arik AVNI, Meitar (IL); Ester ZOHAR, Givataim (IL); Yaron CHEN, Hod Hasharon (IL)

(73) Assignee: MOTORIKA LIMITED, Hamilton (BM)

(21) Appl. No.: 15/774,902

(22) PCT Filed: Nov. 11, 2016
(86) PCT No.: PCT/IB2016/056796

§ 371 Date: May 9, 2018

Related U.S. Application Data
(60) Provisional application No. 62/254,255, filed on Nov. 12, 2015.

(51) Int. Cl. G16H 40/63; A63B 24/00; A61B 5/11; A63B 23/04; A61H 1/02; A61H 3/00

(52) U.S. Cl. CPC G16H 40/63; A63B 24/0087; A61B 5/112; G16H 50/30; A61H 1/0259; A61H 3/008; A63B 23/0464

(57) ABSTRACT

Disclosed are apparatuses and methods for training a patient in moving, by executing a session program comprising a plurality of exercises and the order by which the exercises are to be practiced by the patient. In some embodiments, the apparatus includes a processor configured to: receive results of measurements made during an early stage of training according to the session program, said measurements being indicative of parameters characterizing the moving of the patient; and execute a later stage of the session program based on the results received during the early stage of the training.

The present disclosure is in the field of training patients in moving and walking using robotic a rehabilitation apparatus. The rehabilitation apparatus may be, for example, orthotic rehabilitation apparatus, gait rehabilitation apparatus, or movement rehabilitation apparatus.

Some methods and apparatuses in this field are described in International Patent Application Publication Nos. WO09125397; WO0028927; WO14202767; WO0215819; and WO2004009011.

### SUMMARY

The following lists some examples of inventive concepts disclosed in the disclosure that follows.

### EXAMPLE 1

A computer-implemented method for training a patient in moving, the method comprising:

obtaining a session program for the patient, the session program comprising a plurality of exercises and the order by which they are to be practiced by the patient;

receiving results of measurements made during an early stage of training according to the session program, said measurements being indicative of parameters characterizing the moving of the patient; and

executing a later stage of the session program based on the results received during the early stage of the training.

### EXAMPLE 2

The computer-implemented method of example 1, wherein the session program includes a first exercise; a second exercise; and instructions to execute the first exercise before executing the second exercise, and the method comprising:

executing the first exercise;

during execution of the first exercise, receiving results of measurements indicative of a compliance level of the patient in practicing the first exercise;

and switching to executing the second exercise after the results received indicate a compliance level equal to or higher than a target compliance level.

### EXAMPLE 3

The computer-implemented method of example 1, wherein the session program includes a first exercise; a second exercise; and instructions to execute the first exercise before executing the second exercise, and the method comprising:

executing the second exercise after executing the first exercise;

during execution of the second exercise, receiving results of measurements indicative of a compliance level of the patient in practicing the second exercise;

and switching to executing the first exercise again, after the results received indicate a compliance level lower than a target compliance level.

### EXAMPLE 4

The computer-implemented method of any one of examples 1 to 3, wherein obtaining the session program comprises:

receiving input indicative of at least one of diagnosis of the patient and performance level of the patient; and

generating the session program based on the input received.

### EXAMPLE 5

The computer-implemented method of any one of examples 1 to 4, wherein the session program includes, for each of the plurality of exercises, at least one target compliance level.

### EXAMPLE 6

The computer-implemented method of example 5, wherein receiving results of measurements comprises receiving from sensors configured to sense forces exerted by the patient during the training.

### EXAMPLE 7

The computer-implemented method of any one of examples 1 to 6, wherein the session program includes a plurality of minimal durations, each of the plurality of minimal durations is associated with a corresponding one or more exercises of the plurality of exercises, and the method comprises:

estimating a compliance level of the patient based on results received during execution of an exercise after the exercise is executed for the minimal duration associated with said exercise.

### EXAMPLE 8

An apparatus for training a patient in moving by executing a session program comprising a plurality of exercises and the order by which the exercises are to be practiced by the patient, the apparatus comprising a processor configured to:

receive results of measurements made during an early stage of training according to the session program, said measurements being indicative of parameters characterizing the moving of the patient; and

execute a later stage of the session program based on the results received during the early stage of the training.

### EXAMPLE 9

An apparatus according to example 8, wherein the session program includes a first exercise; a second exercise; and instructions to execute the first exercise before executing the second exercise, and the processor is configured to:

provide the patient instructions to practice the first exercise;

during execution of the first exercise by the patient, receive results of measurements indicative of a compliance level of the patient; and

providing the patient instructions to practice the second exercise after the results received indicate a compliance level equal to or higher than a target compliance level.

### EXAMPLE 10

The apparatus of example 8, wherein the session program includes a first exercise; a second exercise: and instructions to execute the first exercise before executing the second exercise, and the processor is configured to:

provide the patient instructions to practice the second exercise after practicing the first exercise;

during practicing of the second exercise by the patient, receive results of measurements indicative of a compliance level of the patient; and

provide the patient instructions to execute the first exercise again, after the results received indicate a compliance level lower than a target compliance level.

### EXAMPLE 11

The apparatus of any one of examples 8 to 10, wherein the processor is configured to obtain the session program by generating the session program based on input indicative of at least one of diagnosis of the patient and performance level of the patient.

### EXAMPLE 12

The apparatus of any one of examples 8 to 11, wherein the session program includes, for each of the plurality of exercises, at least one target compliance level.

### EXAMPLE 13

The apparatus of example 12, which comprises sensors configured to sense forces exerted by the patient during the training, and the processor is configured to receive the results of measurements from the sensors.

### EXAMPLE 14

The apparatus of any one of examples 8 to 13, wherein the session program includes a plurality of minimal durations, each of the plurality of minimal durations is associated with a corresponding one of the plurality of exercises, and the processor is configured to:

estimate a compliance level of the patient based on results received during execution of an exercise after the exercise is executed for the minimal duration associated with said exercise.

### EXAMPLE 15

An apparatus for training a patient in walking, the apparatus comprising:

a robot configured to move the patient's legs;

a user interface configured to receive input on a diagnosis of the patient and a performance level of the patient; and

a processor programmed to:

receive input indicative of the diagnosis of the patient and performance level of the patient inputted through the user interface, and generate, based on said input, a session program for the patient, the session program comprising a plurality of exercises and the order by which they are to be practiced by the patient; and

control the robot to move the patient's legs according to the session program.

### EXAMPLE 16

The apparatus of example 15, wherein the session program includes, for each of the plurality of exercises, at least one target compliance level.

### EXAMPLE 17

The apparatus of example 16, further comprising sensors, configured to sense forces exerted by the patient's legs during the training and send signals indicative of said forces, and wherein the processor is programmed to:

receive from the sensors input indicative of forces exerted by the patient's legs during the training;

estimate a compliance level for the patient based on the input;

compare the compliance level estimated based on the input with the target compliance levels; and

control the robot based on the results of the comparison.

### EXAMPLE 18

The apparatus of example 17, wherein control the robot based on the results of the comparison comprises continuing with an exercise as long as the estimated performance level is between two target compliance levels, and a predetermined maximum time has not lapsed.

### EXAMPLE 19

The apparatus of example 17, wherein control the robot based on the results of the comparison comprises switching from a current exercise to the next exercise in the session program if a higher of the two target compliance levels is equal to or smaller than the estimated compliance level.

### EXAMPLE 20

The apparatus of example 17, wherein control the robot based on the results of the comparison comprises switching from a current exercise to the preceding exercise in the session program if a lower of the two target compliance levels is larger than the estimated compliance level.

### EXAMPLE 21

The apparatus of example 17, wherein the processor is programmed to compare the compliance level estimated based on the input with the target compliance levels once in a predetermined time period.

### EXAMPLE 22

The apparatus of example 21, wherein the session program comprises, for each exercise, the predetermined time period.

### EXAMPLE 23

A computer-implemented method of training a patient in walking using a robot configured to move the patient's legs, the method comprising:

receiving, by a processor, input indicative of a diagnosis of the patient and input indicative of performance level of the patient;

generating by the processor, based on said inputs, a session program for the patient, the session program comprising a plurality of exercises and the order by which they are to be practiced by the patient; and

controlling the robot to move the patient's legs according to the session program.

### EXAMPLE 24

The method of example 23, wherein the session program includes, for each of the plurality of exercises, at least one target compliance level.

### EXAMPLE 25

The method of example 24, further comprising: receiving, by the processor input indicative of forces exerted by the patient's legs during the training, said receiving being from sensors configured to sense said forces;

estimating a compliance level for the patient based on the input;

comparing the compliance level estimated based on the input with the at least one target compliance level; and

controlling the robot based on the results of the comparison.

### EXAMPLE 26

The method of example 25, wherein controlling the robot based on the results of the comparison comprises continuing with an exercise as long as the estimated compliance level is between two target compliance levels, and a predetermined maximum time has not lapsed.

### EXAMPLE 27

The method of example 25, wherein controlling the robot based on the results of the comparison comprises switching from a current exercise to the next exercise in the session program when the estimated compliance level is above a target compliance level.

### EXAMPLE 28

The method of example 25, wherein controlling the robot based on the results of the comparison comprises switching from a current exercise to a preceding exercise in the session program if the estimated compliance level is below a target compliance level.

### EXAMPLE 29

The method of example 25, wherein the processor is programmed to compare the compliance level estimated based on the input with the at least one target compliance level once in a predetermined time period.

### EXAMPLE 30

The method of example 29, wherein the session program comprises, for each exercise, the predetermined time period.

### EXAMPLE 31

An apparatus for training a patient in walking, the apparatus comprising a processor configured to:

generate a session program for the patient, the session program comprising a plurality of exercises, an order by which the exercises are to be practiced by the patient during the session, and at least one compliance target for each exercise:

cause displaying of instructions to the patient to practice according to the session program;

receiving input from sensors sensing reactions of the patient to the instructions displayed; and

cause providing feedback to the patient during the session, said feedback being indicative of the patient's compliance with the instructions in comparison with the at least one target compliance level.

### EXAMPLE 32

The apparatus of example 31, further comprising a display configured to display the instructions to the patient during training, the display comprising:

an input for receiving data from the processor; and

at least one screen or loudspeaker for displaying the instructions to the user based on data received from the processor.

### EXAMPLE 33

The apparatus of example 31 or 32, further comprising the sensors configured to sense reactions of the patient to the instructions displayed.

### EXAMPLE 34

The apparatus of any one of examples 31 to 33, further comprising a user interface, and wherein the processor is configured to receive from the user interface input indicative of a diagnosis of the patient and a performance level of the patient, and generate the session program based on said input.

### EXAMPLE 35

The apparatus of any one of examples 31 to 34, wherein the processor is configured to:

receive data indicative of performance of the patient in a set of exercises: and generate the session program based on said data indicative of performance of the patient in the set of exercises.

### EXAMPLE 36

The apparatus of example 35, wherein the processor is configured to determine a performance level of the patient based on said data indicative of performance of the patient in a set of predetermined exercises.

### EXAMPLE 37

The apparatus of any one of examples 31 to 36, further comprising a hoist to carry a portion of a weight of the patient when the patient carries out the exercises, and said session program comprises for at least one exercise the portion of the weight of the patient carried by the hoist.

### EXAMPLE 38

The apparatus of example 37, wherein the processor is configured to control the hoist to carry said portion of the weight of the patient.

### EXAMPLE 39

The apparatus of any one of examples 31 to 38, further comprising a treadmill, and said session program comprises for at least one exercise a speed for the treadmill.

### EXAMPLE 40

The apparatus of example 39, wherein the processor is configured to control the speed of the treadmill according to the session program.

### EXAMPLE 41

The apparatus of any one of examples 31 to 40, further comprising a robotic arm configured to connect to a leg of the patient, and the processor is configured to control the robotic arm according to the session program.

### EXAMPLE 42

The apparatus of any one of examples 31 to 41, wherein the processor is configured to modify the session program based on input received from the sensors during the execution of the session.

### EXAMPLE 43

A computer-implemented method of training a patient in walking according to a session program, the method comprising:

executing a computer-program that generates a session program for the patient based on a diagnosis of the patient and a performance level of the patient, the session program comprising a plurality of exercises, an order by which the exercises are to be practiced by the patient during the session, and at least one compliance target for each exercise;

displaying instructions to the patient to carry out the session program;

receiving input from sensors sensing reactions of the patient to the instructions displayed; and

providing feedback to the patient during the session, said feedback being indicative of the patient's compliance in comparison with the at least one compliance target.

### EXAMPLE 44

The computer-implemented method of example 43, wherein said providing is by controlling a view on a screen, a loudspeaker, or both.

### EXAMPLE 45

The computer-implemented method of example 43 or 44, wherein said providing comprises causing the patient to move differently than before the feedback is provided.

### EXAMPLE 46

The computer-implemented method of example 43 or example 44, comprising receiving the diagnosis of the patient and a performance level of the patient through a user interface.

### EXAMPLE 47

The computer-implemented method of any one of examples 43 to 45, comprising:

receiving data indicative of performance of the patient in a set of exercises; and generating the session program based on said data indicative of performance of the patient in the set of exercises.

### EXAMPLE 48

The computer-implemented method of example 47, comprising determining a performance level of the patient based on said data indicative of performance of the patient in a set of predetermined exercises.

### EXAMPLE 49

The computer-implemented method of any one of examples 43 to 48, further comprising controlling a hoist to carry a portion of a weight of the patient when the patient carries out the exercises, said controlling of the hoist being according to the session program.

### EXAMPLE 50

The computer-implemented method of any one of examples 43 to 49, wherein at least one exercise included in the session program includes walking on a treadmill, and said session program comprises, for at least one exercise that includes walking on the treadmill, a speed for the treadmill.

### EXAMPLE 51

The computer-implemented method of any one of examples 43 to 50, comprising controlling a robotic arm according to the session program, said robotic arm being configured to connect to a leg of the patient so as to move the leg of the patient.

### EXAMPLE 52

computer-implemented method of any one of examples 43 to 51, further comprising modifying the session program based on the input from the sensors.

### EXAMPLE 53

An apparatus for training a patient in practicing a particular gait event, the apparatus comprising:

a robot configured to move the patient's legs;

a processor configured to control the robot to move the patient's legs so as to produce gait cycles;

sensors, configured to sense forces exerted by the patient's legs during the training and send signals indicative of said forces; and

a display, configured to display instructions to the patient when the robot moves the patient's legs,

wherein the processor is further configured to:

when the robot moves the legs of the patient through the particular gait event, send signals to the display to instruct the patient to act, and

adjust the control of the robot based on signals sent from the sensor, said signals being indicative of the reaction of the patient to the instructions displayed on the display when the robot moves the legs of the patient through the particular gait event.

### EXAMPLE 54

The apparatus of example 53, further comprising a user interface configured to allow a user to indicate the particular gait event, and the processor is configured to determine, based on input from the user interface, the particular gait event.

### EXAMPLE 55

The apparatus of example 53 or example 54, wherein the particular gait event is selected from a group consisting of: heel-strike, support, toe-off, leg-lift, and swing.

### EXAMPLE 56

The apparatus of any one of examples 53 to 55, wherein the processor is configured to adjust the control of the robot if the action of the patient is outside a compliance range, and keep the control of the robot unchanged if the action of the patient is inside said compliance range.

### EXAMPLE 57

The apparatus of any one of examples 53 to 56, wherein the processor is configured to adjust the control of the robot to move the patient's legs faster than before the patient was instructed to act, if the action of the patient is inside a compliance range.

### EXAMPLE 58

The apparatus of example 56 or 57, wherein the processor is configured to determine if the patient's action is outside or inside said compliance range based on signals sent from the sensors.

### EXAMPLE 59

The apparatus of any one of examples 53 to 58, wherein the display comprises at least one of a visual display and an auditory display.

### EXAMPLE 60

A computer-implemented method for training a patient in performing a particular gait event, the method comprising:

controlling a robot to move the patient's legs so as to produce gait cycles; instructing the patient to act when the robot is controlled to move the patient's legs to perform the particular gait event; and

adjusting the control of the robot based on actions made by the patient after the patient is instructed to act.

### EXAMPLE 61

The computer-implemented method of example 60, comprising:

determining a compliance level of the actions made by the patient after the patient is instructed to act, based on input from sensors, said input being indicative of forces exerted by the patient's legs; and

adjusting the control of the robot based on the determined compliance level.

### EXAMPLE 62

The computer-implemented method of example 60 or example 61, further comprising receiving from a user interface an indication as to which gait event is to be the particular gait event, and controlling the robot based on said indication.

### EXAMPLE 63

The computer-implemented method of any one of examples 60 to 62, wherein the particular gait event is selected from a group consisting of: heel-strike, support, toe-off, leg-lift, and swing.

### EXAMPLE 64

The computer-implemented method of example 61, wherein adjusting the control of the robot comprises:

adjusting to move the patient's legs slower than before the patient was instructed to act if the determined compliance level is outside a compliance range, and keeping the control of the robot unchanged if the determined compliance level is inside said compliance range.

### EXAMPLE 65

The computer-implemented method of example 61, wherein adjusting the control of the robot comprises:

adjusting to move the patient's legs faster than before the patient was stimulated to act if the determined compliance level is inside a compliance range.

### EXAMPLE 66

An apparatus for training a patient in performing a particular gait event, the apparatus comprising:

at least one processor configured to:

determine a gait event to be trained;

identify a gait event of a patient; and

instruct the patient to act based on comparison between the gait event identified and the gait event determined.

### EXAMPLE 67

An apparatus according to example 66, wherein the at least one processor is configured to receive from at least one sensor data indicative of the gait event of the patient, and identify the gait event of the patient based on the data received from the at least one sensor.

### EXAMPLE 68

An apparatus according to example 66 or 67, comprising at least one sensor that senses forces exerted by legs of the patient, and wherein said at least one processor is configured to receive data from said at least one sensor and identify the gait event of the patient based on said data.

### EXAMPLE 69

An apparatus according to any one of examples 66 to 68, comprising a robotic arm configured to connect to a leg of the patient and move the leg of the patient, and the at least one processor is configured to control the robotic arm to move the leg of the patient in a gait cycle comprising a plurality of cycle points.

### EXAMPLE 70

An apparatus according to example 69, wherein the at least one 5 processor is configured to identity a gait event of the patient based on the cycle points through which the leg of the patient is moved.

### EXAMPLE 71

An apparatus according to any one of examples 66 to 70, comprising a display, configured to display instructions to the patient while the patient is training, and wherein the at least one processor is configured to instruct the patient by displaying instructions on the display.

### EXAMPLE 72

An apparatus according to any one of examples 66 to 71, comprising a user interface allowing a user to communicate with the at least one processor, wherein the at least one processor is configured to determine the gait event to be trained based on input received via the user interface.

### EXAMPLE 73

An apparatus according to any one of examples 66 to 72, wherein the at least one processor is configured to receive data indicative of forces exerted by a leg of the patient along a gait cycle, and analyze said data to determine the gait event to be trained.

### EXAMPLE 74

An apparatus according to any one of examples 66 to 73, wherein the at least one processor is configured to adjust a control of a robotic arm configured to connect to a leg of the patient and move the leg of the patient, said adjust of control being based on signals sent from at least one sensor that senses forces exerted by legs of the patient, said signals being indicative of a reaction of the patient to instructions provided to the patient by the at least one processor based on comparison between the gait event identified and the gait event determined.

### EXAMPLE 75

A computer-implemented method of training a patient in walking using a robot configured to move legs of the patient so as to produce walking cycles, the method comprising:

measuring a first force applied by a leg of the patient when the patient is instructed to be relaxed and the leg is moved by the robot.

measuring a second force applied by the leg of the patient when the patient is instructed to move the leg; and

taking an action based on a net force, said net force being a difference between the second force and the first force, said taking an action comprising one or more of:

instructing the robot to move a leg of the patient;

instructing the patient to move his leg; and

providing real-time feedback to the patient regarding compliance of a performance of the patient with a target performance.

### EXAMPLE 76

The computer-implemented method of example 75, wherein each of the first force and second force is measured when the patient carries a same portion of a weight of the patient.

### EXAMPLE 77

The computer-implemented method of example 75 or 76, comprising: receiving, from a user through a user interface, data indicative of said same portion of the weight of the patient, and controlling a hoist to lift the patient so that all the weight of the patient but said portion is carried by the hoist.

### EXAMPLE 78

The computer-implemented method of any one of examples 75 to 77, comprising taking the action at a late point along a gait cycle based on net force measured at an early point along the walking cycle wherein going through the gait cycle comprises going first through the early point and thereafter through the late point.

### EXAMPLE 79

The computer-implemented method of example 78, wherein taking the action comprises instructing the robot to slow down at the later point if the net force measured at the early point is below a threshold.

### EXAMPLE 80

The computer-implemented method of example 78 or 79, wherein taking the action comprises instructing the robot to speed up at the later point if the net force measured at the early point is above a threshold.

### EXAMPLE 81

The computer-implemented method of any one of examples 75 to 80, wherein taking the action comprises moving a leg of the patient.

### EXAMPLE 82

The computer-implemented method of any one of examples 75 to 81, wherein taking the action comprises instructing the patient to move.

### EXAMPLE 83

The computer-implemented method of any one of examples 75 to 82, wherein taking the action comprises providing real-time feedback to the patient regarding compliance of a performance of the patient with a target performance.

### EXAMPLE 84

An apparatus for training a patient in walking, the apparatus comprising:

a robot configured to move legs of the patient so as to produce gait cycles;

a sensor configured to sense forces applied by a leg of the patient; and

a processor configured to:
receive from the sensor signals indicative of forces applied by the leg of the patient;
distinguish between signals of a first kind and signals of a second kind, wherein the signals of the first kind are signals received from the sensor when the patient is instructed to be relaxed and the leg is moved by the robot, and signals of the second kind are signals received from the sensor when the patient is instructed to move the leg;
determine a net force as a difference between a force indicated by the signals of the first kind and a force indicated by the signals of the second type; and
take an action based on the net force determined.

The action may include one or more of:

moving the leg of the patient;

instructing the patient to move his leg; and

providing real-time feedback to the patient regarding compliance of a performance of the patient with a target performance.

### EXAMPLE 85

The apparatus of example 84, wherein the processor is configured to:

operate a display to instruct the patient to relax, and identify signals received when the display is operated to instruct the patient to relax as signals of the first kind, and

operate the display to instruct the patient to walk actively, and identify signals received when the display is operated to instruct the patient to walk actively as signals of the second kind.

### EXAMPLE 86

The apparatus of example 84 or 85, wherein the processor is configured to:

receive from a user interface a first indication that a passive walking begins and identify signals received from the sensor after receiving said first indication as signals of the first kind; and

receive from a user interface a second indication that an active walking begins and identify signals received from the sensor after receiving said second indication as signals of the second kind.

### EXAMPLE 87

The apparatus of any one of examples 84 to 86, further comprising a hoist, and the processor is configured to control the hoist to lift the patient so as to reduce weight of the patient that rests on the patient's legs.

### EXAMPLE 88

The apparatus of any one of examples 84 to 87, wherein the 10 processor is configured to:

instruct the robot to move the leg of the patient at a late point along a gait cycle based on net force determined at an early point along the gait cycle wherein going through the gait cycle comprises going first through the early point and thereafter through the late point.

### EXAMPLE 89

The apparatus of example 88, wherein the processor is configured to instruct the robot to slow down at the late point if the net force measured at the early point is below a threshold.

### EXAMPLE 90

The apparatus of example 88 or 89, wherein the processor is configured to instruct the robot to speed up at the late point if the net force determined at the early point is above a threshold.

### EXAMPLE 91

The apparatus of any one of examples 84 to 90, wherein the action comprises moving the leg of the patient.

### EXAMPLE 92

The computer-implemented method of any one of examples 84 to 91, wherein the action comprises instructing the patient to move.

### EXAMPLE 93

The computer-implemented method of any one of examples 84 to 92, wherein the action comprises providing real-time feedback to the patient regarding compliance of a performance of the patient with a target performance.

### EXAMPLE 94

A computer-implemented method for training a patient in walking, the method comprising:

controlling a hoist to lift the patient so that the entire body weight of the patient is carried by the hoist:

controlling a robot to move the patient's legs so as to produce gait cycles without touching the ground;

receiving from sensors results of measurements of forces exerted by the patient's legs during the walking cycles without touching the ground;

controlling the hoist to lower the patient so that at least part of the body weight of the patient is carried by the patient's legs; and

based on the measurements received when the entire body weight of the patient was carried by the hoist, controlling the robot to move the patient's legs so as to produce gait cycles when at least part of the body weight of the patient is carried by the patient's legs.

### EXAMPLE 95

The computer-implemented method of example 94, further comprising instructing the patient to be relaxed and not to exert any force on the robot when producing walking cycles without touching the ground.

### EXAMPLE 96

The computer-implemented method of example 95, wherein said instructing comprises displaying instructions to the patient using at least one of an audial or visual display.

### EXAMPLE 97

An apparatus for training a patient in walking, the apparatus comprising:

a robot configured to move the patient's legs, the robot comprising a plurality of motors, each configured to move a respective part of a patient leg; and

a processor configured to:
control the robot to move the patient's legs so as to walk through a gait cycle;
receive data indicative of forces exerted by each of the motors to move the patient's legs through the gait cycle; and
control the display to present data indicative of forces exerted by each of the motors independently of forces exerted by the other motors.

### EXAMPLE 98

The apparatus of example 97, wherein for each of the motors, the data indicative of forces exerted by the motor comprises data indicative of currents consumed by the motor.

### EXAMPLE 99

The apparatus of example 97 or 98, wherein the processor is configured to control the display in real time, so that during each instant, the forces being presented by the display are the forces being exerted by the motors.

### EXAMPLE 100

The apparatus of any one of examples 97 to 99, wherein the data is presented by an image of a human leg, and data indicative of forces exerted by a motor that moves a part of a leg of the patient is presented by coloring the respective part of the leg in the image, so that different colors represent different ranges of forces.

### EXAMPLE 101

The apparatus of example 100, wherein the processor is configured to control the display in real time, and parts of the image of the leg move in accordance with the gait cycle.

### EXAMPLE 102

The apparatus of any one of examples 97 to 101, wherein the data is presented by presenting a figure comprising a plurality of parts colored with different colors, each part being associated with a respective portion of a gait cycle, and each color representing a different difference between measured forces and reference forces.

### EXAMPLE 103

A method of training a patient in walking using a robot configured to move the patient's legs, the robot comprising a plurality of motors, each configured to move a respective part of a patient leg, the method comprising:

controlling the robot to move the patient's legs so as to walk through a gait cycle;

receiving data indicative of forces exerted by each of the motors to move the patient's legs through the gait cycle; and

controlling a display to present the received data, so that forces exerted by each of the motors is presented independently of forces exerted by the other motors.

### EXAMPLE 104

The method of example 103, wherein for each one of the motors, the data indicative of forces exerted by the motor comprises data indicative of currents consumed by the motor.

### EXAMPLE 105

The method of any one of examples 103 to 104, wherein controlling the display is in real time, so that during each instant, the forces being presented by the display are the forces being exerted by the motors.

### EXAMPLE 106

The method of any one of examples 103 to 105, wherein the data is presented by an image of a human leg, and data indicative of forces exerted by a motor that moves a part of a leg of the patient is presented by coloring the respective part of the leg in the image, so that different colors represent different ranges of forces.

### EXAMPLE 107

The method of example 106, wherein controlling the display is in real time, and parts of the image of the leg move in accordance with the gait cycle.

### EXAMPLE 108

The method of any one of examples 103 to 107, wherein the data S is presented by presenting a figure comprising a plurality of parts colored with different colors, each part being associated with a respective portion of a gait cycle, and each color representing a different difference between measured forces and reference forces.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the an how embodiments of the invention may be practiced.

FIG. 1A is a block diagram of an apparatus for training a patient in walking according to some embodiments of the invention;

FIG. 1B is a diagrammatic representation of a gait rehabilitation apparatus according to some embodiments of the invention, and a zoom-in view a portion of the device according to some embodiments of the invention;

FIG. 2 is a flowchart of a method of training a patient in performing a particular gait event according to some embodiments of the invention;

FIG. 3 is a block diagram of an apparatus for generating program sessions for training a patient in walking according to some embodiments of the invention:

FIG. 4 is a flowchart of a method of obtaining and executing a training session program according to some embodiments of the invention;

FIG. 5 is a flowchart of a method of running a training session for training a patient in walking according to some embodiments of the invention:

FIG. 6 is a flowchart of a method of training a patient in walking using a robotic orthotic or gait rehabilitation apparatus, according to some embodiments of the invention;

FIG. 7 is a flowchart of a method of training a patient in walking using a robotic orthotic apparatus, according to some embodiments of the invention;

FIG. 8 is a block diagram describing a training apparatus according to some embodiments of the invention.

The present disclosure is in the field of training patients in walking using robotic gait rehabilitation apparatus. The patients typically suffer from neurological conditions or orthopedic injuries. Example of neurological conditions may include head injury, post-stroke condition, and Parkinson disease. Examples of orthopedic injuries may include total hip replacement, total knee replacement, and total ankle replacement.

Some embodiments of the present invention include methods and apparatuses for personalized training of patients, using integration of clinical rehabilitation principles, knowledge, and Rules. For example, the disclosed methods and apparatuses allow to Initiate passive movement slowly to normalize muscle tone and arrive to selected active muscle movement which allows detection of patient active ability.

In some embodiments, the disclosed methods and apparatuses may detect gait deviation-weight bearing asymmetry, gait abnormal pattern (heel to toe), stance/swing asymmetry, step size asymmetry and detect actual functional ability, also referred herein as performance level.

In some embodiments, the actual functional ability/performance level, in combination with diagnosis of the patient, may set an optimal gait training program (also referred herein as session program). The program may include a combination of various modes of training, for example, passive mode, active mode with or without biofeedback, focused training of specific gait events, etc.

In some embodiments, a real time compliance score is measured during the session, based on a combination of a objective parameters, such as weight balance symmetry, resistance, and active participation. Each such parameter may have a different weight and according to the weighted score and its difference from a target score the system may decide to move forward or backward in executing the session program.

Some embodiments of the invention allows training patients based on a fast initial objective evaluation of parameters such as patient function ability, gait pattern, weight bearing, comfortable speed, active ability, foot placement, and resistance. The evaluated parameters may be correlated to standard functional ability tests and allow machine functional score.

Thus, in some embodiments, a session program for training a patient may be generated, executed, and modified based on measurements taken during the execution. The session program may include a set of exercises to be practiced by the patient during the session, the order of their execution along the session, and some targets, with which the patient is to comply in order to continue progressing along the session according to the program. If the patient does not meet the compliance targets, he may be required to go back to a preceding exercise.

Some embodiments of the invention include the generation of the session program, for example, based on knowledge of a diagnosis of the patient, and the patient's performance level (functional ability). In some embodiments, the functional ability itself is measured by an apparatus according to the present invention, based on performance levels shown by the patient in exercises that are found to correlate with standard tests for determining functional ability. In some embodiments, the session program is generated based on parameters measured in order to determine the functional ability of the patient, instead of, or in combination with, the functional ability. These parameters may include, for example, symmetry between weights carried by each side of the patient's body (also known as weight bearing symmetry), symmetry in force applied to load cells at the two hips of the patient, comfortable walking speed, and symmetry between step sizes taken in right and left leg.

Some embodiments of the invention include methods and apparatuses for calibrating measurements of forces applied by a leg of the patient during gaiting, and acting based on the calibrated forces.

In some embodiments, the calibration includes measuring a first force, applied by the patient non-intentionally, for example, when the patient is relaxed and moved only by the robot. Such movement by the robot alone is referred to herein as passive walking. The calibration may also include measuring a second force, applied by the patient intentionally, when the patient is actively engaged in walking. Such movement by the patient actively participating in moving the legs is referred to herein as active walking. Finally, the calibration may include subtracting the force measured to be applied during passive walking from the forces measured to be applied during active walking, to obtain net force.

In some embodiments, only a portion of the patient's weight is carried by the patient during walking, and the rest of the weight is carried by a hoist. In some embodiments, the portion of the weight carried by the patient during passive walking is the same as the portion carried by the patient during active walking. This kind of calibration may provide enhanced sensitivity to the force measurements, and the actions taken based on the net force so obtained may be more effective than if obtained based on the force measured during active walking alone.

In some embodiments, the portion of the weight carried by the patient (or by the hoist) may be provided to the processor from a user interface. Optionally or additionally, that portion may be a parameter characterizing an exercise in a session program.

In some embodiments, actions are taken based on the net force. For example, a certain instruction may be displayed to the patient when the net force is above some predetermined threshold: a certain instruction may be provided to robotic arms of the robot and/or to a treadmill of the robot based on the net force; and/or a certain feedback may be provided ot the patient based on the net force. The feedback may include signs that the patient complies (or does not comply, as the case may be) with a target compliance level. The instruction to the patient may be to apply more force at a certain point along the gait cycle (e.g., where it is identified that the net force is too low if the patient is not explicitly instructed to be more active at that point). The instructions to the robot may be to walk more slowly, for example, if the net force is below a target threshold.

In some embodiments, the apparatus may include a sensor, for sensing force applied by the patient during walking, and a processor configured to receive signals indicative of the forces sensed by the sensor. The processor may further be contigured to control the robot and a display. The robot may be configured to move the patient's legs, and the display may be configured to provide instructions and/or feedback to the patient. In some embodiments, the processor may be configured to distinguish between signals received from the sensor during passive walking and active walking; calculate the net force based on a difference between forces applied during active walking and forces applied during passive walking, and control the robot and/or the display based on the net force.

The present disclosure also refers to gait rehabilitation apparatuses and methods specifically configured for training different particular gait events. A gait cycle of a person may be considered to include several gait events, for example, heel strike, toe-off, and swing. A patient may have particular difficulty in one of them, and in such cases the presently disclosed apparatuses and methods may be advantageous in providing training focused on the performance of that particular gait event.

In some embodiments, a therapist may identify a gait event requiring specific training. The therapist may then instruct the apparatus to train this gait event particularly. The instruction may be provided via a user interface, configured to receive such instructions. The user interface may be connected to a processor configured to control the apparatus based on input received from the user interface.

In some embodiments, a gait event requiring specific training may be identified by the gait rehabilitation apparatus. The apparatus may then indicate to the therapist, e.g., via the above-mentioned (or other) user interface, that a need is identified for special training of the particular gait event. In some embodiments, the therapist may decide if to train the patient focusing on the particular gait event, or when to start such training. In some embodiments, the processor starts training the patient focusing on that gait event, unless the therapist instructs otherwise.

Identification of the particular gait event that requires focused training may be obtained by analyzing results of measurements taken during a regular use of the apparatus by the patient. For example, the apparatus may include sensors attached to the feet of the patient, and these sensors may provide data on forces exerted by different parts of each foot. This data may be analyzed to find abnormality in a particular one of the gait event.

In some embodiments, the specific training may include an alert to the patient that the particular gait event is to begin. Such alert may cause the patient to pay more attention to his actions when training this particular gait event. In some embodiments, the specific training may include instructing the patient to be more active (or begin being active) when the particular gait event begins. Being more active may include, for example, exerting more force.

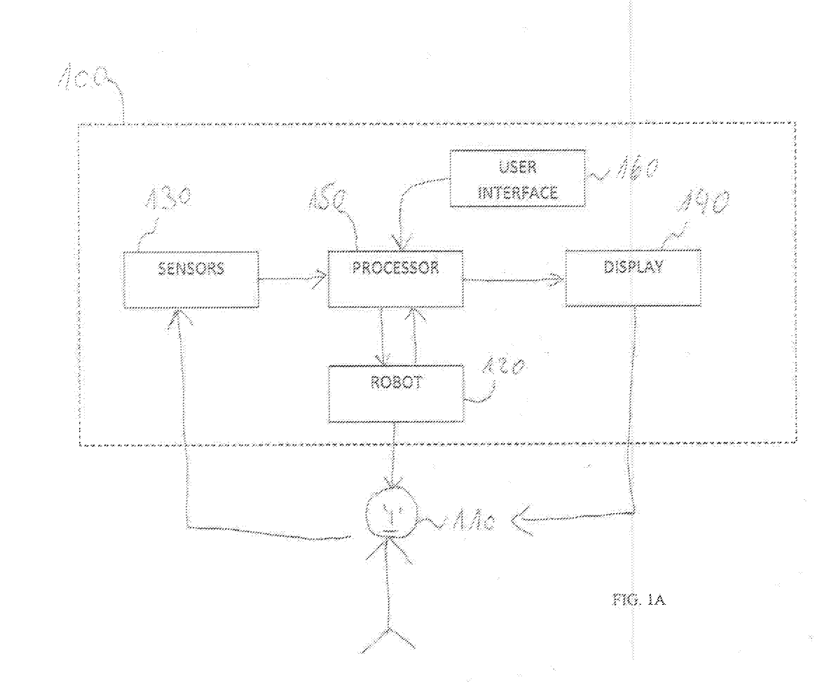

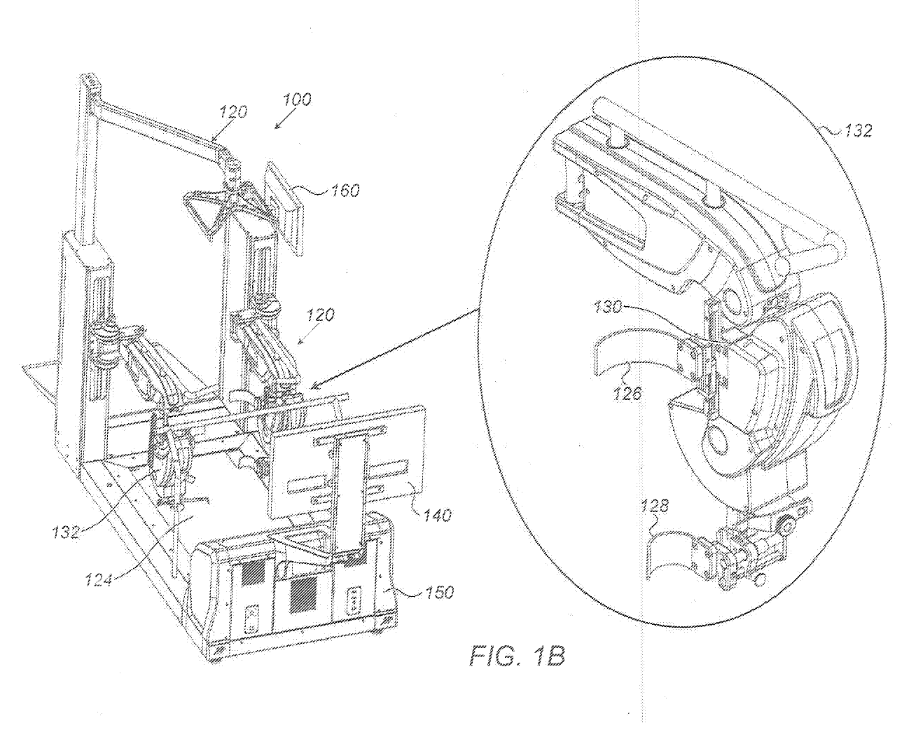

FIG. 1A is a block diagram describing an apparatus 100 for training a patient 110 in walking. Apparatus 100 is shown to include a robot 120, sensors 130, a display 140, and a processor 150. FIG. 1B is a diagrammatic presentation of apparatus 100.

The robot 120 is configured to move the legs of the patient, for example, when a portion of the weight of the patient is carried by a hoist 122. In some embodiments, apparatus 100 may also include a treadmill (124), on which the patient can walk, for example, when some of the patient's weight is carried by hoist 122 and/or when the legs of the patient are moved by robot 120. To move the legs of the patient, the robot 120 may include leg cuffs (126, 128) designed to wrap a leg (e.g., at the thigh, below the knee, and/or near the ankle). The cuffs may be connected to robotic arms 132 of robot 120. Each of the robotic arms may be connected to a motor or any other arrangement that can move the robotic arms in a controlled manner. Movement of the robotic arms of robot 120 may be controlled by processor 150, and the robot may send feedback to the processor as to the position of the cuffs in real time, so the processor may have information of where the cuffs are in practice, and not only to where they should have been moved.

Sensors 130 may include, in some embodiments, load cells at the hips of the patient. Sensors 130 may include, additionally or alternatively to sensors at the hips, sensors at the knees (e.g., below the knee) at the ankles (e.g., right above the ankle), in the sole of a shoe of the patient, etc. In some embodiments, sensors 130 may include one or more weight sensors, sensing the weight that the hoist carries. This weight may indicate the weight of the patient, if the patient is lifted off the ground, or the weight of the patient carried by the patient himself, which may be calculated as a difference between the weight of the patient and the weight carried by the hoist. In some embodiments, sensors 130 may include sensors that sense how much weight is carried at each side of the hoist. Such sensors may allow estimating how much weight is carried by each leg of the patient. Sensors 130 may sense, for example, forces exerted by patient 110 on one or more of the cuffs, for example, on each of two hip cuffs 126. In some embodiments, sensors 130 may sense both magnitude of forces and direction of forces. In some embodiments, the measurements made by the sensors may indicate muscles activity of the patient (e.g., power and direction of action), or any other parameter that may be indicative to the activity of the muscles that move the legs of the patient, also referred herein as leg muscles. Sensors 130 may include sensors installed in or near the cuffs, for example, where the cuff touches the patient or his cloths, near the connection between the cuff and the robotic arm, etc. In some embodiments, sensors 130 may include sensors positioned at the patient's foot (e.g., in the sole of the patient's shoe). Sensors 130 may be configured to send signals indicative of the sensed forces or parameters characterizing them to processor 150. Sensors 130 may sense actions of patient 110 and send respective signals in real time, that is, when the patient is training in walking using the robot. Data indicative of the sensed signals may be transmitted from the sensors to the processor directly, or via intermediate one or more devices that receive the data and transfer them to the processor, as received or after some processing. The communication between sensors 130 and processor 150 may be wired, wireless, or may be wired along some portion or portions of the way and wireless along other portion(s) of the way.

In some embodiments, the processor may be on a remote server (e.g., in a public or private cloud providing apparatus 100 cloud computing services). The data may be sent to the remote server via a communication network (e.g., the Internet), analyzed at the server, and the analysis results may be sent back through the communication network to apparatus 100. In some embodiments, the analysis results (whether analyzed remotely or locally) may include instructions to the robot to move in one way or another, for example, faster or slower. Optionally or alternatively, the analysis results may include instructions to a display (e.g., display 140), to display to the patient exercising-instructions selected by the server for the patient based on the measurement results. These instructions may be designed, in some embodiments, to train the patient in practicing a specific gait event. In some embodiments, the anadlysis results may include recommendations for the therapist, and the therapist may decide if to accept them, accept them in some amended form, or reject them. For example, a recommendation of the server may include recommendation to train the patient in performing heel strike using a particular exercise, and the therapist may accept the recommendation, decide on training the patient in performing heel strike using another exercise, or reject the recommendation. In some embodiments, the therapist may decide to delay his decision about the recommendation, e.g., using a snooze-like function.

In some embodiments, the analysis, whether performed remotely or locally, may include analysis of net force. The net force may be the force exerted by the patient during training minus the force exerted by the patient when the patient is relaxed and his legs are moved by the robot. This may make the analysis more sensitive to changes in the force exerted intentionally by the patient, because the use of net force allows ignoring forces independent of the patient's intentional efforts, e.g., the weight of the legs.

Working through the cloud may allow, for example, loading new exercises centrally to different apparatuses connected to the same cloud. This way, if a new exercise is found to be clinically useful, the cloud may be loaded with this exercise. In some embodiments, the cloud may be further loaded with rules when to apply or suggest the new exercise. This way, the new exercise is made available to users of all similar apparatuses connected to the same cloud. Working through the cloud may also be advantageous in that therapists may provide input and feedback on different exercises and their efficacy in different clinical situations, and this information may be shared with all other users on the fly. Alternatively or additionally, the information inputted by the users may be used to improve the recommendations provided by the cloud. In some embodiments, the clinical efficacy of exercises may be estimated by the cloud, based on ongoing changes in the data received from the patients, and improve the recommendations best on such estimations. Although the term cloud is used, the invention is not limited to any particular service provision architecture, and may utilize, for example, one or more dedicated servers.

Processor 150 may be configured to control robot 120 to move the legs of patient 110 so as to produce gait cycles.

As used herein, the term “processor” may include an electric circuit that performs a logic operation on input or inputs. For example, such a processor may include one or more integrated circuits, microchips, microcontrollers, microprocessors, all or part of a central processing unit (CPU), graphics processing unit (GPU), digital signal processors (DSP), field-programmable gate array (FPGA) or other circuit suitable for executing instructions or performing logic operations.

The instructions executed by the processor may, for example, be pre-loaded into the processor or may be stored in a separate memory unit such as a RAM, a ROM, a hard disk, an optical disk, a magnetic medium, a flash memory, other permanent, fixed, or volatile memory, or any other mechanism capable of storing instructions for the processor. The processor(s) may be customized for a particular use, or can be configured for general-purpose use and can perform different functions by executing different software.

In some embodiments, more than one processor is employed to execute one or more recited instructions. This is emphasized by reference to “at least one processor”, but any processor recited herein may be replaced with a plurality of processors that together are configured to execute the recited instructions. In such embodiments, all employed processors may be of similar construction, or they may be of differing constructions. The employed processors may be electrically connected or disconnected from each other. They may be separate circuits or integrated in a single circuit. When more than one processor is used, they may be configured to operate independently or collaboratively. They may be coupled electrically, magnetically, optically, acoustically, mechanically or by other means permitting them to interact.

As used herein, if a structure (e.g., a robot, a processor, etc.) is described as being “configured to” perform a particular task (e.g., configured to move a patient's leg), then the structure includes components, parts, or aspects (e.g., software) that enable the machine to perform the particular task. In some embodiments, the structure performs this task during operation. For example, a processor configured to perform a task may be programmed to execute instructions that together result in the performance of the task.

Each gait cycle may include gait events that together compose steps. Examples to such events (also referred to as phases) may include: heel-strike, support, toe-off, leg-lift, and swing. In the heel-strike phase, the foot hits the ground heel first. After the heel strike phase, the leading leg hits the ground, and the muscles work to cope with the force passing through the leg. This is known as the support phase. In the toe-off phase, the foot prepares to leave the ground—heel first, toes last. Once the foot has left the ground, the lower limb is raised in preparation for the swing phase. This is known as the leg-lift phase. In the swing phase, the raised leg is propelled forward. This is where the forward motion of the walk occurs. Next, the heel hits the ground, and the whole cycle repeats. In some embodiments, the gait cycle may be divided to gait events differently, for example, to a stance phase, push-off phase, and swing phase. Another possible division of the gait cycle is to stance phase and swing phase only. Another possible division of the gait cycle is to six phases: heal strike, loading response, mid-stance, terminal-stance, pre-swing, initial and mid-swing, and terminal swing. The invention does not depend on the specific way in which the gait cycle is divided to phases or events. The robot walks the patient through all the phases, and the sensors continuously transmit data indicative of forces applied by the patient, so the processor can combine input from the robotic arms or their control with input from the sensors to tell what forces are applied by the patient at each gait event.

In some embodiments, processor 150 is configured to move robot 120 (or its arms) through a large number of cycle points along the gait cycle, e.g., through 50, 100, 200, 360, or any smaller, larger or intermediate number of cycle points. The cycle points may be distributed at equal time-differences along a gait cycle. The walking pace may be set by setting the size of the time difference between the cycle points: the longer it takes to move from one cycle point to the next, the slower is the walking pace. The robot may go through these cycle points fluently, so a fluent movement is produced. The processor may include a memory that stores correspondence between cycle points along the gait cycle and gait events. This way, the processor may identify a gait event of a practicing patient at any moment by the cycle point through which the robot goes at that moment. Processor 150 may instruct display 140 to display an instruction to patient 110 based on the cycle point through which the robot goes, and this way, synchronize between the instructions provided to the patient and the patient's current gait event.

In some embodiments, processor 150 may instruct display 140 to display online feedback to the patient. In some embodiments, the online feedback may be indicative of forces, e.g., net forces, exerted by the patient. In some embodiments, the online feedback may be indicative of the compliance of the patient with the instructions provided. The compliance may indicate to the patient how close is the force exerted to a target force. For example, if a target net force of 2 kg was set for the patient, and the patient exerts net force of only 2 kg or more, the display may show a sign to the fact that the patient's achievement is in compliance with the target. Such a mark may include, for example, green footmarks displayed on a screen in synchronization with the patient's walking. If the net force exerted by the patient is smaller than 2 kg, the display may show a sign to the fact that the patient's achievement is not in compliance with the target. Such a mark may include, for example, red footmarks displayed on a screen in synchronization with the patient's walking. The footmarks may be shown to move in the pace and step-size of the patient, to provide the patient feedback on these parameters in addition to the feedback on the compliance with the target force exertion. If one leg (e.g., the right leg) exerts 2 kg force or more, while the other leg exerts less than 2 kg, the display may show right footmarks in green and left footmarks in red. This is an example to foot-specific feedback that may be provided by the processor through the display, so that the patient can concentrate his efforts at the leg that is not yet in compliance with the target, and be pleased with the performance of the other leg.

Processor 150 may provide similar online feedback through channels other than (or additional to) display 140. For example, the online feedback may be in the form of change in the walking pace.

In one such example, if the exerted force (e.g., in both legs) is below a target threshold, the processor may control the robot to slow down the patient's gaiting, and if the target threshold is not reached, for example, within a predetermined time period, stop the gaiting, for example, to let the patient rest. In some embodiments, a compliance threshold may be set. In some embodiments, the compliance threshold may be set in terms of an average of achievements in both legs. The compliance threshold may also take into account additional factors, e.g., a symmetry between the lengths of the steps taken by both legs, symmetry (or differences) between weight carried by each leg, etc.

In another one such example, if a compliance threshold is reached (e.g., the exerted force is above a target threshold), the processor may control the robot to speed up the walking pace, so as to train the patient in faster walking. In both examples the pace change (either slow down or speed up, as the case may be) provides online feedback to the patient indicative of the patient's compliance.

In some embodiments, processor 150 may be configured to instruct display 140 to display a predetermined instruction to patient 110 based on real time user input. For example, the apparatus may include a user interface configured to receive from a user (e.g., a therapist) indication that practicing a particular gait event should now take place. In one such example, the user interface may include a “practice now” button, which the therapist may push when the therapist sees that the patient enters the gait event to be practiced. In some embodiments, in immediate response to the button being pushed, processor 150 instructs display 140 to act, e.g., to show or otherwise display instructions to the patient. The processor may further follow the compliance of the patient with the instructions, adjust further instructions, and adjust control of the robot based on the compliance. In some such embodiments, the processor may use the therapist input to learn when a gait event starts. For example, the user interface may further allow the user to indicate which gait event is going to be practiced, and the processor may be configured to associate the indicated gait event with cycle points, through which the robot moves the patient's legs when the user pushes the “practice now” button. This association mechanism may be used, for example, to “teach” processor 150 identifying a gait event. In some embodiments, the association mechanism may be used to allow a therapist to define to apparatus 100 new gait events.

An aspect of some embodiments of the invention may be processor 150 as such, or any gait rehabilitation apparatus comprising it. In some such embodiments, processor 150 may be configured to determine a gait event to be trained. As explained above, the determination which gait event to train may be based on user input. In some embodiments, the determination may be based on analysis, optionally performed by processor 150, of data received from sensors 130.

In some embodiments, processor 150 may be configured to identify a gait event of a patient, for example, as explained above, using the cycle points through which robot 120 goes. Alternatively or additionally, processor 150 may use input from sensors 130 to identify a gait event. Alternatively or additionally, processor 150 may use online user input to identify a gait event.

In some embodiments processor 150 may be configured to instruct the patient to act based on comparison between the gait event identified and the gait event determined to require focused training. Processor 150 may instruct the patient by causing specific instructions to be displayed on display 140. The instructions may be displayed, for example, audibly, visually, and/or textually.

In some embodiments, processor 150 is configured to receive from sensors 130 data indicative of the gait event of the patient. For example, sensors at the sole may provide data indicative of hill strike step stage being practiced. The processor may be configured, in some embodiments, to identify the gait event of the patient based on the data received from the at least one sensor. Once identified, the gait event may be compared with the gait event determined to require focused training, and training may continue accordingly.

In operation, display 140 may display instructions to patient 110 while the patient is training, for example, the display may display an instruction to apply forces so as to follow the robot, so that part of the force moving the leg is exerted by the patient, and only the remainder of the force is exerted by the robot. The instructions may be displayed textually, visually, audibly, or by any combination of two or more of text, audio and video.

In some embodiments, processor 150 may be configured to control display 140 to instruct patient 110 to act when the robot moves the legs of the patient through the particular gait event. Sensors 130 may sense the actions made by patient 110, and send respective signals to processor 150. Processor 150 may be configured to adjust the control of robot 120 based on signals indicative of the actions the patient made following the display of the instructions on display 140.

In some embodiments, apparatus 100 may include a user interface 160 configured to allow a user to indicate the particular gait event, during which the patient is to be instructed to act. The user interface may include a touch screen, keypad, optical reader (e.g., for reading barcodes or QR codes), or any other means useful for receiving input from a user. Processor 150 may be configured to determine the particular gait event based on input from the user interface, and control the display accordingly. In some embodiments, the robot may also be controlled based on input received from the user interface.

For example, in some embodiments, processor 150 may be configured to adjust the control of the robot if the action of patient 110 is below or above a compliance threshold, or outside a compliance range defined between two compliance thresholds. The compliance threshold may be, for example, a value of sensed parameters, a value of ratios between sensed parameters, or a ratio between a value of a sensed parameter and a target value of the same parameter, or any other value indicative of the patient compliance with the instructions provided to him by display 140. Such values may include size of force exerted by the patient, direction of the force, timing of the force exertion, etc. Preferably, the force may by the net force, obtained by subtracting force exerted when the patient is relaxed and moved by the robot alone, from force exerted during active walking. Optionally, the force may be the force measured during training, without such subtraction. In one example, in an exercise where the patient is required to respond to instructions, a compliance indicator may be calculated based on a success rate e.g., the portion of the instructions, to which the patient responded within a predefined time period from receiving the instruction. This portion (as well as other compliance indicators) may be used to evaluate a compliance level. In another example, when a patient is required to increase his walking speed from time to time, a compliance indicator may be calculated based on the average walking speed, divided by a target average walking speed. In another example, when the robotic arms are not in use, e.g., when the patient walks on a treadmill, partly lift by the hoist or independently of the hoist, a ratio between step sizes (and/or stepping speed) in both legs may be a compliance indicator. For example, equal step size may give the highest value to the compliance indicator, and the compliance indicator may decrease in value as the difference (or ratio) between step size in the two legs increases. In another example, the length of the step size, e.g., in comparison with a target step size may be used as a compliance indicator. In some embodiments, a compliance level may be an average of values of two or more compliance indicators. In some embodiments, the average may be a weighted average, with different weights assigned to different compliance indicators. In some embodiments, the weights may be equal.

The adjustment of the control of robot 120 may be designed to provide motoric feedback to patient 110 on his compliance. For example, in some embodiments, if the compliance of the patient is below an acceptable compliance threshold the robot may slow down and keep slowing down until it stops, unless the compliance of the patient improves during the slowing down. If the compliance is above the threshold to start with, no slowing down will be experienced by the patient. If the robot stops, the robot may provide the patient some predetermined time off and then begin the exercise again.

The exercise may begin with the robot walking the patient through all the gait events in a regular gait for some steps, and then instructing the patient to exert forces during a particular gait event as described above.

In some embodiments, the patient may be instructed to exert forces continually, and strengthen the force exerted when so instructed via display 140. If successful (e.g., if the compliance is above a threshold), the robot may be controlled to, walk the patient at higher speed.

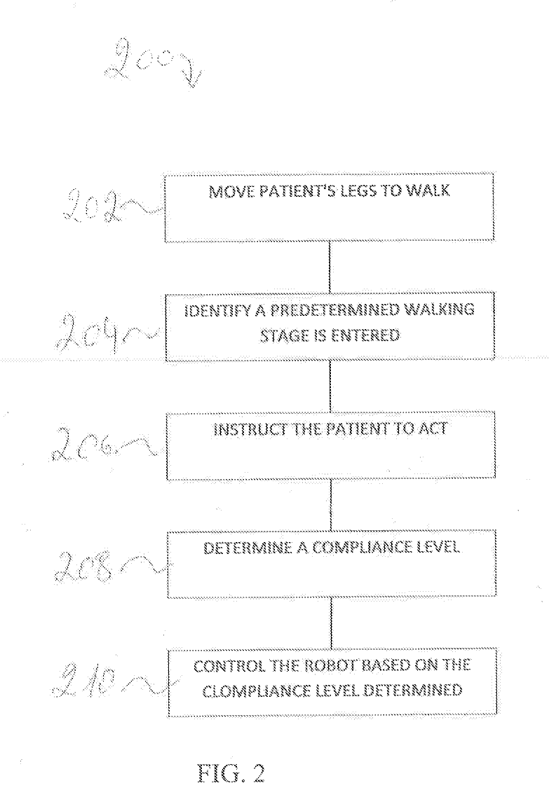

FIG. 2 is a flowchart of actions to be taken in carrying out a method 200 according to some embodiments of the invention. Method 200 may be computer-implemented, and in particular, may be implemented by processor 150 of apparatus 100 shown in FIGS. 1A and 1B. The computer implementing method 200 may be local to apparatus 100 or remote, for example, dedicated to controlling gait rehabilitation devices, or on a cloud. Method 200 may be useful for training a patient in performing a particular gait event. Gait events are described above.

In 202 a robot (e.g., robot 120) may be controlled to move the patient's legs so as to produce gait cycles.

In 204 it is identified that the patient is entering the particular gait event that has to be trained. The identification may be carried out as described above.

In 206 the patient is instructed (e.g., by appropriately controlling display 140) to act. This step is performed when it is identified that the patient is entering, or about to enter the gait event that has to be trained. The instruction to act may be displayed to the patient synchronously with the patient's entrance to the particular gait event (e.g., at a cycle point before, during, or shortly after starting the particular gait event). The processor may receive data indicative of the particular gait event that is to be trained from a user interface, e.g., from user interface 160 described above. In some embodiments, method 200 may include receiving data indicative of the gait pattern of the patient. These data may include measurements of forces exerted by the foot on the ground (e.g., what part touches, at what force, and when). Such data may be obtained in some embodiments from sensors sensing forces applied by (or on) the patient's foot, for example, sensors inside a shoe of a patient, for example, on or below a sloe of the shoe. In such embodiments, the processor may use this data to conclude that a particular gait event is to be trained, and what this particular gait event is. In some embodiments, the processor may suggest a therapist to train this particular gait event. In some embodiments, the processor may start training this particular gait event without receiving explicit instructions from the therapist to do so. For example, in some embodiments, a therapist may be able to provide the processor general instructions to train specific gait event whenever the processor finds this adequate. In some embodiments, the therapist may require that the processor waits explicit instructions before starting training a patient in a particular gait event. In 206, the control of robot 120 may be adjusted based on actions made by the patient after the patient is instructed (e.g., via display 140) to act.

In some embodiments, step 208 may include determining a compliance level of the actions made by the patient after step 206 was taken. The compliance level may be determined based on input received from sensors (e.g., sensors 130), indicative of the forces exerted by the patient in response to the instructions the patient received in step 204.

In 210 the control of the robot is adjusted based on the determined compliance level. For example, the robot may be controlled to move the patient's legs slower than before step 206 was taken, if the determined compliance level is below a compliance threshold, and keeping the control of the robot unchanged if the determined compliance level is equal to or above the compliance threshold.

In another example (or in addition to the previous example), step 210 may include adjusting the control of the robot to move the patient's legs faster than before step 206 was taken if the determined compliance level is above a compliance threshold.

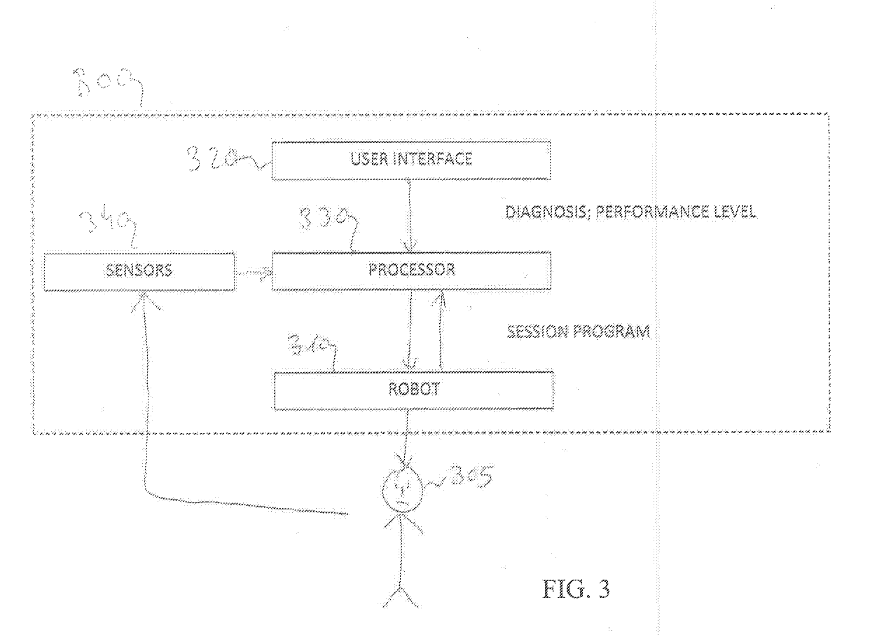

FIG. 3 is a block diagram of an apparatus 300 for training a patient in walking. Apparatus 300 includes a robot 310 configured to move legs of patient 305; a user interface 320; and a processor 330. User interface 320 is configured to receive input on a diagnosis of the patient and a performance level of the patient. The input may be put in by a therapist. The diagnosis may be selected by the therapist from a list of conditions that apparatus 300 may be useful in treating. The performance level of the patient may also be inserted by the therapist, for example, based on past experience with the patient, tests performed before using apparatus 300, and the therapist clinical impression from the patient. Apparatus 300 may also have a memory, saving personal data on the patient, such as name, gender, age, etc.

In some embodiments, the performance level of the patient may be one of predetermined performance levels, going, for example, from requiring maxima support to independent. For example, a patient that can walk on a treadmill without any help of the robotic arms may have an “independent” performance level. This may include patients that a portion of their body weight is supported by the hoist during training. In another example, a patient that requires the hoist to support his entire body weight, and can hardly exert forces intentionally in response to stimulations, may be considered “require maximal support”. Patients in the middle between these two states may be considered, for example, requires some support, and requires considerable support. In some embodiments there are four performance levels, but the invention is not limited to any particular number of performance levels.

In some embodiments, input indicative of the performance level of the patient may include data, from which the processor may arrive at the performance level. For example, in some embodiments, the patient may be required to carry out a standard set of exercises, and the performance of the patient during execution of these exercises may be evaluated by a skilled therapist to conclude the performance level of the patient. Such standard exercises may include, for example, the Berg balance test, timed up to go test, and 10 m walk test.

In some embodiments, the set of standard, known in the art exercises, may be replaced with a set of predetermined exercises performed on an apparatus according to an embodiment of the present disclosure (e.g., apparatus 100 or apparatus 300). Clinical trials may be held to verify correlation between performance levels indicated by performance on an apparatus according to the present disclosure, and performance levels indicated by existing standard tests.

Processor 330 may be configured to receive via user interface 320 input indicative of the diagnosis of the patient and performance level of the patient, and generate a session program for the patient based on the input.

A session program is a program for a training session. A training session is a single occasion, during the patient practices a plurality of exercises, which may include walking exercises. A session may start with connecting the patient to the apparatus, and may end with disconnecting the patient from the apparatus. The connection may include, for example, connection to the hoist or connection to a leg cuff. In some embodiments, during a session, the patient may be disconnected from a leg cuff, but stay connected to the hoist. In some embodiments, the duration of a training session is about an hour, although shorter or longer sessions are not excluded from embodiments of the invention. For example, if a patient is very weak, he may execute a short session of about 15 minutes or 20 minutes. If a patient is quite strong, he may practice sometimes even for longer than hour, for example, 70 minutes or 90 minutes. In many cases, however, a session takes between 45 minutes and 60 minutes.

Some exemplary parameters that may be taken into consideration for generating a session program include: symmetry between weights carried by each side of the patient's body (also known as weight bearing symmetry), symmetry in force applied to load cells at the two hips of the patient, comfortable walking speed, and symmetry between step sizes taken in right and left leg.

The session program may include a plurality of exercises and the order by which they are to be practiced by the patient. In some embodiments, processor 330 may include a memory storing an association-generating code, e.g., a lookup table, associating each pair of diagnosis and performance level with a session program. The associating code may be prepared based on clinical experience gained with a similar apparatus, where the session programs are decided by human therapists, rather than by the processor. Processor 150 may be further configured to control the robot to move the patient's legs according to the session program.

Each of the exercises may be characterized, for example, by exercise parameters. Examples of exercise parameters may include pace of exercising, step length, gait event to practice, minimal time to practice before the patient's compliance is evaluated, maximal time to devote to the exercise, a minimum compliance threshold, a maximum compliance threshold, etc. Different exercises may have different parameters, for example, some exercises may be made to train a particular gait event, and some not, so the parameter “gait event to train” is not relevant to all exercises.

In some embodiments, the exercises may be characterized by modes. For example, in a first mode the patient may be expected to be completely passive, and the legs of the patient are moved just by the robot. Exercise parameters in this exercising mode may include the duration of the exercise, the speed of walking, step length, portion of patient body weight supported by the patient, body weight supported by the patient, etc. Working in this exercising mode may be used for setting a baseline for forces measured in other modes. For example, the forces exerted on the load cell at the hip during exercising in this mode may be subtracted from forces exerted on the same load cell at the same hip when exercising in another mode.

In a second mode, the patient may be expected to exert force only in response to a stimulation (e.g., instruction given via a display). In this mode the exercise parameters may include, in addition to duration, speed, and step length, for example, a duration before the first stimulation, a duration before first estimation of patient's compliance, the duration for which the robot waits for the reaction of the patient to the stimulation, etc.

In a third mode, the patient may be expected to walk when some of the force is applied by the robot, and part by the patient himself, and the patient should increase the force when stimulated to do so. Some exercise parameters additional to those useful in the second mode may be: how much force is applied by the robot between periods of increased force by the patient.

In a fourth mode, the patient may walk by himself (e.g., on a treadmill), and the exercise parameters may be, for example, speed of walking, portion of body weight supported by the patient, and possibly other exercises the patient has to practice during walking. The invention is not limited to a particular set of modes and exercise parameters characterizing the exercises composing the session programs.

In some embodiments, in addition to exercising parameters as described above, each exercise may be characterized by a target compliance level. As used herein a compliance level may be any parameter indicating the quality of performance of a patient in carrying out an exercise. The compliance level may include a value of one or more parameters, each indicating an aspect of the performance quality. In some embodiments, a compliance level is an average of several such parameters. The average may be weighted, so that each parameter may have its own weight. In some embodiments, some of the weights or all the weights are equal. A compliance level may be evaluated considering values of one or more parameters, for example, a portion of the training time, where the patient exerts forces unnecessarily (e.g., in the mode where the patient is expected to be completely passive), ratio between step size in one leg and step size in the other leg (e.g., in the mode where the patient walks on a treadmill free of the robotic arms), how long does it take to the patient to react to a stimulation, how effective (e.g., strong, well-directed) are the forces that the patient exerts responsive to the stimulation (e.g., size and direction of the forces), etc. A target compliance level may be a value of a compliance level, which the patient is expected to reach or exceed. In some embodiments there may be two target compliance levels (also referred herein as compliance thresholds or target compliance thresholds): a minimum one, which the patient is expected to reach or exceed, and a maximal one, which if exceeded, it may be indicative to a need to replace the exercise with a more challenging one.

In some embodiments, the program session determined by processor 150 includes a target compliance level for at least one of the exercises, for example, for all the exercises.

In some embodiments, apparatus 300 further includes sensors 340 that sense forces exerted by the patient during the training. Processor 330 is configured, in some such embodiments, to receive from the sensors input indicative of forces sensed by the sensors. Processor 330 may be configured to associate a compliance level to the patient's actual performance during training. The processor may be configured to make such an association based on the input received from sensors 34). In some such embodiments, processor 330 may be configured to compare the compliance level associated to the patient's actual compliance to target compliance levels making part of the program session. The program session may have been determined by the processor based on the data received via user interface 310 (e.g., diagnosis). Processor 330 may be further configured to control the robot based on results of the comparison. For example, if the compliance level is above a predetermined threshold, the processor may stop the current exercise, and start the next exercise in the session. In some embodiments, one or more of the exercises in a program session includes a high target compliance level and a low target compliance level, and if the patient does not reach the low target compliance level, the processor stops the exercise, and begins the preceding exercise once again. If the patient reaches the high target compliance level, the processor may stop the current exercise and begin the next exercise in the session. In some embodiments, if the compliance level of the patient is between the high and low target compliance levels, the current exercise is continued, for example, to a predetermined time, after which the patient's performance level may be compared again with the target compliance levels.

In some embodiments, processor 330 is configured to compare the compliance level estimated based on the input from sensors 340 with the target compliance levels once in a predetermined time period. In some embodiments, the session program comprises, for each exercise, the predetermined time period.

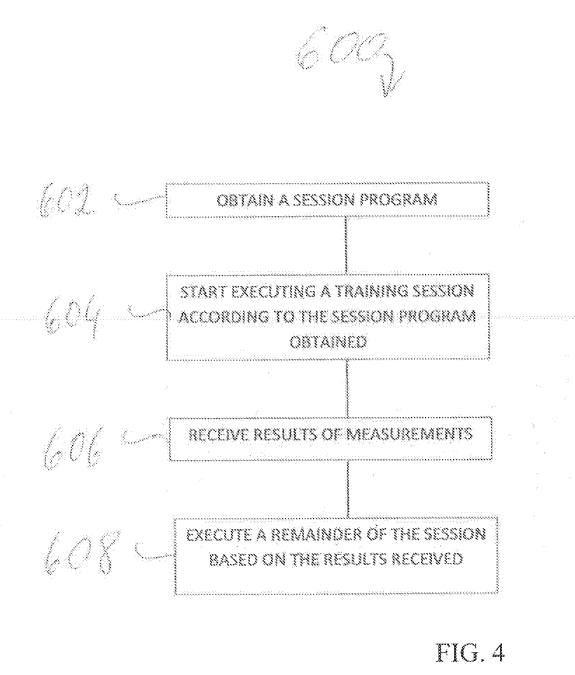

FIG. 4 is a flowchart of a method 600 of training a patient in moving, according to some embodiments of the invention. The moving may include, for example, walking, and/or moving the hands of the patient.

Method 600 may include a step 602 of obtaining a session program for the patient. In some embodiments, the session program may be obtained from an external source, e.g., from a remote memory via a communication link or network (e.g., via the Internet). In some embodiments, the session program may be generated locally or remotely, e.g., based on input from a user. The input may be inputted via a user interface, e.g., user interface 320. The input may include at least one of a diagnosis of the patient, and a performance level of the patient, e.g., as estimated by a therapist, or as deduced from measurements taken before method 600 begins. The session program may include a plurality of exercises and the order by which they are to be practiced by the patient.

Method 600 may further include a step 604 of starting to execute a training session according to the session program obtained.

Method 600 may further include a step 606 of receiving results of measurements made during an early stage of the execution of the training session (e.g., during step 604). The results may be received (directly or indirectly) from sensors, e.g., sensors 340. The measurements may be indicative of parameters characterizing the moving of the patient. For example, in case the moving comprises walking, the parameters may include step size in each leg, forces (e.g., net forces) exerted by the patient's legs, etc. An exercise may be considered executed in “early” or “late” stage in the training in accordance with the time at which it is executed. For example, an exercise executed first makes part of an earlier stage of the training than an exercise that is being executed last in the session. Thus, measurements results obtained at a certain time may be taken into account in a later time during the same session.

Method 600 may further include a step 608 of executing a later stage of the session program based on the results received during the early stage of the training. For example, executing the remainder of the session (after execution of step 602), based on the results obtained.

For example, the session program may include a first exercise; a second exercise; and instructions to execute the first exercise before executing the second exercise. In some embodiments, method 600 includes executing the first exercise first: and during execution of the first exercise, receiving results of measurements indicative of a compliance level of the patient in practicing the first exercise. Then, the compliance level of the patient may be estimated based on the measurement results, and compared to a target compliance level. In some embodiments, the target compliance level makes part of the obtained session program. The method may include switching from executing the first exercise to executing the second exercise only after the estimated compliance level is equal to or higher than a target compliance level.

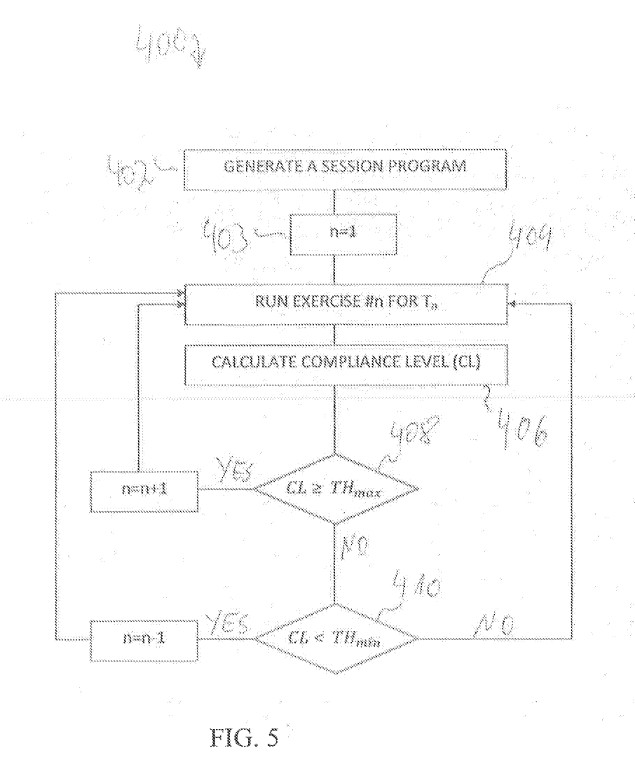

Similarly, in some embodiments, method 600 includes executing the first exercise first; and then the second exercise. During execution of the second exercise, results of measurements indicative of a performance level of the patient in practicing the second exercise are received. Then, the compliance level of the patient may be estimated based on the measurement results, and compared to a target compliance level associated with the second exercise. In some embodiments, the target compliance levels and their association to the different exercises taking part in the session makes part of the session program obtained in step 602. The method may include switching from executing the second exercise back to executing the first exercise again, if the estimated compliance level is lower than a target compliance level. These examples are explained in some more detail in reference to FIG. 5, described below.

In some embodiments, the session program includes, for each of the exercises included in the session program, a minimal duration. Each exercise may be executed for the minimal duration before the compliance level of the patient is being estimated. In some embodiments, after a compliance level is estimated, and the same exercise continues, the compliance level may be estimated again after another period of the same length. In some embodiments, the minimal duration before the first estimate of patient compliance level may be different (e.g., longer) than a duration between later estimates. In some embodiments, the period between each two subsequent estimations of the patient's performance level may differ. For example, this duration may be determined by the compliance level estimated for the patient. For example, if the compliance level is quite far from the target, longer time may lapse before the compliance level is estimated again, than if the patient's compliance level is very close to the target.

The method of FIG. 4 and that of FIG. 5 may be carried out, for example, by an apparatus as described in FIGS. 1A, 1B, and 3, wherein the processor is configured to carry out the respective method.

FIG. 5 is a flowchart of a computer-implemented method 400 for running a training session for training a patient in walking using a rehabilitation robot, according to some embodiments of the invention.

In step 402, a session program is received or generated. The session program may be generated online by the computer or generated in advance, e.g., by a therapist, and communicated to the computer, e.g., via a user interface. The session program includes identification of exercises, the order by which the exercises are to be performed. Each exercise may also include a minimum compliance threshold and a maximum compliance threshold.

In step 403 the serial number n of the exercise to be executed is set to 1.

In step 404, the patient executes an exercise of the serial number n. Executing the exercise may include active leg manipulation by the robot (e.g., robot 120). In some embodiments, the computer controls the robot to execute the exercise. Step 404 may be carried out for a minimal time Tn, which may be a parameter of exercise #n in the session program.

In step 406, after the exercise is being run for the minimal time, a compliance level (CL) is calculated based on data received from the sensors.

In step 408, the calculated compliance level is compared with the maximum compliance threshold (THmax) provided in the session program. If the calculated compliance level is equal to or larger than the maximum compliance threshold (408: YES), the serial number of the exercise to be executed is enlarged by 1, and the method continues to step 404 (unless there is no further exercise in the session, in which case the session ends). If the calculated compliance level is below the maximum compliance threshold (408: NO), the method goes to step 410.

In step 410, the calculated compliance levels are compared with the minimum compliance threshold provided in the session program. In some embodiments, if the calculated compliance level is under the minimal compliance threshold, (410: NO), n is decreased by one, and the method goes back to step 404, that is, the session goes back to the preceding exercise. However, if n=1 (not shown), and there is no easier exercise in the session program, an alert is sent to the therapist, to indicate that the patient does not reach his goals even in the first exercise. In some embodiments, instead of alerting the therapist or in addition to such an alert, a new session program is generated, but for a patient with a compliance level lower by one degree from the compliance level for which the session program was originally generated. If the calculated compliance level is between the minimum and maximum thresholds (410: YES), the program returns to step 404, to run the same exercise for an additional minimum runtime.

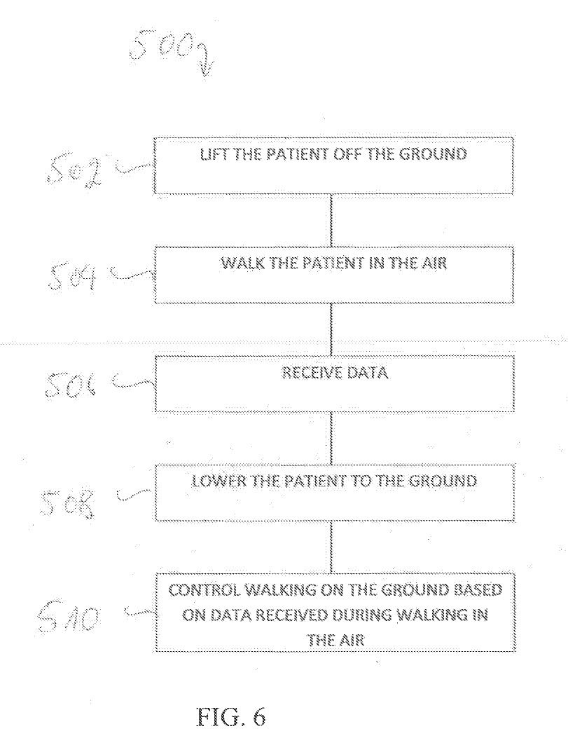

FIG. 6 is a flowchart of a computer-implemented method 500 for training a patient in walking using a robotic orthotic or gait rehabilitation apparatus, according to some embodiments of the invention. Method 500 includes step 502 of controlling a hoist to lift the patient so that the entire body weight of the patient is carried by the hoist. This may allow training the patient in making walking steps without carrying in the same time any part of the patient's body weight. Such an exercise may be referred herein as walking in the air. In walking in the air training, the patient may be instructed to be completely relaxed. The instructions may be provided, for example, via a display displaying instructions to the patient during training. The display may display the instruction by voice, visual effect, and/or text. Forces exerted by the patient may include forces attributable to spasticity of the patient. Change of forces attributable to spasticity of the patient may indicate progress of the training. For example, decrease of forces attributable to spasticity during a training session may indicate that the spasticity of the patient was improved during the session. Similarly, decrease or eventual elimination of forces attributable to spasticity during some time period comprising a plurality of training sessions may indicate that the spasticity of the patient improved (either thanks to the training sessions, or other treatment the patient received in parallel, e.g., by medication).

The forces exerted by each leg when the patient did not carry any of his weight on his own legs may be indicative of an effective weight of the respective leg. The effective weight may include force required to balance gravitational force acting on the leg and, if the patient is spastic, force required to balance the spasticity.

In some embodiments, measurements carried out when the patient did not carry any of his weight on his own legs may be used as a baseline for later measurements, when weight is carried by the patient. For example, a patient may be instructed to actively. Such instructions may be provided, for example, when the entire weight of the patient is carried by the hoist, or when some of the weight of the patient is still carried by the hoist, and some is carried by the patient himself. The effective weight of the leg is not affected by the effort of the patient to participate in the walking. Thus, to evaluate the net force intentionally exerted by the patient on a leg, the effective weight of the leg may be subtracted from the force measured to be applied by the leg to the leg cuff. e.g., by a load cell near the hip. Further training may be controlled based on the net force.

Method 500 may further include step 504 of controlling a robot to move the patient's legs so as to produce walking in the air cycles.

Method 500 may further include step 506 of receiving from sensors (e.g., sensors 130 or 340) results of measurements of forces exerted by the patient's legs during walking in the air.

Method 500 may further include a step 508 of controlling the hoist to lower the patient so that at least part of the body weight of the patient is carried by the patient's legs. Such walking may be referred to herein as walking on the ground. In some embodiment, walking on the ground may be carried out when the patient is on a treadmill, so that the treadmill may assist in setting a walking speed for the patient.

Method 500 may further include step 510 of controlling the robot to walk the patient on the ground. In some embodiments, the controlling of step 510 may be based on measurements received from the sensors when the patient walked in the air. For example, a program session may be determined for a patient based on comparison of results obtained in two different events of walking in the air. Optionally or additionally, a program session may be determined for a patient based on net forces applied during a walking on the ground exercise.

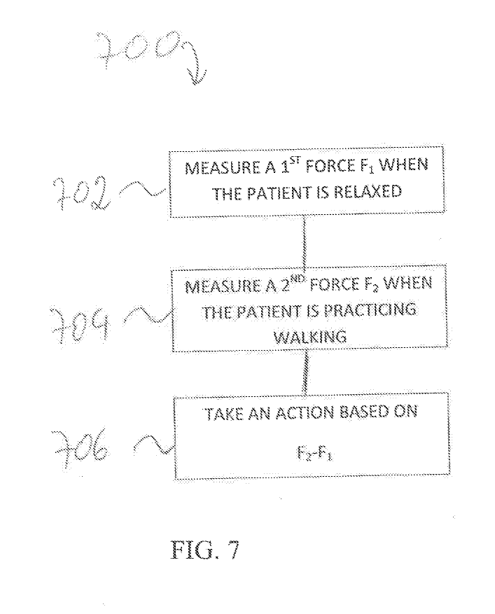

FIG. 7 is a flowchart of a method 700 of training a patient in walking using a robot configured to move legs of the patient so as to produce walking cycles. Method 700 may be computer-implemented, for example, it may be implemented by processor 150 of FIGS. 1A and 1B or processor 330 of FIG. 3. Method 700 includes steps of measuring a first force and a second force.

In step 702, the first force is measured. e.g., by sensors 130 or 340, when the patient (e.g., patient 305) is instructed to be relaxed and let his legs being moved by the robot (i.e., to be engaged in passive walking). In some embodiments, the first force may be measured when the entire weight of the patient, or a portion of the weight of the patient, is carried by a hoist (e.g., hoist 120).

In step 704, the second force is measured, e.g., by the same sensors measured the first force, when the patient is instructed to move the legs by his own, or together with the robot (i.e., to be engaged in active walking). In some embodiments, the second force may be measured when the same portion of the weight of the patient is carried by the hoist as during the passive walking. For example, the passive and active walking may be done when all the weight is on the hoist, or when 20%, 25%, 30%, 50%, or any other fraction of the weight is carried by the patient himself.

Method 700 may further include a step 706 of acting based on the net force, defined as a difference between the second force and the first force. Acting based on the net force may include one or more of: instructing the robot to move the leg of the patient based on the net force; providing the patient real-time feedback based on the net force; and instructing the patient to act based on the net force. In some embodiments, real time feedback may include any feedback that the patient perceives as if it is provided to him at the same time he is performing the action that triggers the feedback. In practice, there may be a time difference of up to about 0.1, 0.2, or 0.25 seconds between the patient's action and the feedback he receives on the same action.

In some embodiments, method 700 may include measuring the first and second forces at each of a plurality of gait cycle points, and determining a net force (e.g., by calculation) for each gait cycle point as a difference between second and first forces measured at that gait cycle point. Step 706 may then include acting differently at different gait cycle points. For example, step 706 may include acting based on a value indicative of the net forces measured at different gait cycle points. Such a value may be, for example, an average over all the points, a value indicative of changes in the net forces along the gait cycle, e.g., one or more parameters of a function describing the net force as a function of gait cycle point. For example, if the net force changes periodically, the parameters may include an amplitude value a frequency value, and/or an amplitude value of a trigonometric function (e.g., sine or cosine) that best fits the periodic change of the net force. The phase may be indicative of a gait cycle point at which the net force is at maximum (and/or a gait cycle point at which the force is at minimum).

Step 706 of instructing the robot to move according to the net force may include, in some embodiments, instructing the robot to move differently at different points along gait cycles. For example, in some embodiments, a gait event to be trained may be identified based on the net force measured at some gait cycle points, and in step 706 the robot may train the patient in performing this gait event in a more focused manner. Identification of a gait event to be trained may be based, for example, on a drop of net force that occurs whenever the patient enters this gait event.

Step 706 of providing the patient real-time feedback based on the net force may include, in some embodiments, showing to the patient on the display (e.g., display 140) an indication to a compliance level, indicative to the extent by which the patient complies with target values predefined for the net forces. The compliance level may include, for example, a difference (or ratio) between an average net force, and a target net force. In addition to showing the feedback on the display, in some embodiments, providing the feedback may include changing the pace of walking by controlling the robotic arms and/or the treadmill. For example, if a compliance level is above a threshold, providing the feedback may include speeding up the patient's walking. It is noted that in such a case, providing the feedback may be by means of instructing the robot to move differently than before.

Step 706 of instructing the patient to move based on the net force may include in some embodiments instructing the patient to go faster or slower, e.g., based on a compliance level being above or below a threshold as discussed above. In some embodiments, instructing the patient to move based on the net force may include instructing the patient to act upon entrance of a particular gait event.

In some embodiments, step 706 may be carried out at the same gait cycle as step 704. For example, the robot and/or patient is instructed to move at a late point in a gait cycle based on net force measured at or calculate for an early point in the very same gait cycle. That is, the adaptation of the robot behavior to the net force may take place during the very same gait cycle. A point in a gait cycle is referred to as “early” and “earlier” if the patient (and/or robot) goes through this point before going through a point referred to as “late” or “later”. In other words, the “late” and “early” descriptors are given based on the order of appearance in a gait cycle or in a training session.

In some embodiments, step 706 may be carried out not at the same gait cycle at which the net force was determined, but at a later gait cycle in the same exercise during the same training session.

In some embodiments, method 700 may be practiced using apparatus 100 of FIGS. 1A and 1B and/or apparatus 300 of FIG. 3, if they are appropriately configured, e.g., by programming.

An apparatus 100 or 300 configured to carry out method 700 may include: a robot 120, configured to move legs of the patient so as to produce gait cycles; a sensor 130, configured to sense a force applied by a leg of the patient when the leg of the patient moves; and a processor 150.

Processor 150 may be configured to: receive from sensor 130 signals indicative of forces applied by the leg of the patient; and distinguish between first signals, received from the sensor when the patient is instructed to be relaxed and the leg is moved by the robot, and second signals, received from the sensor 130 when the patient is instructed to move the leg. The first and second signals may be referred to herein as signals of first and second kinds, respectively.

For example, apparatus 100 or 300 may include a user interface 160, configured to allow a user to indicate when the patient is instructed to walk passively. In one such embodiment, user interface 160 may include a “calibration” button. The user (e.g., therapist) may instruct the patient to be relaxed, and push the calibration button, e.g., when the user believes the patient is indeed relaxed. Processor 150 or 330 may be configured to identify signals received after the calibration button is pressed as signals of the first kind. After the patient gaited for some cycles, the user may push a “start training” button, and instruct the patient to start active walking. The processor may be configured to identify signals measured after the “start training” button is pushed as signals of the second kind.

In some embodiments, whenever a training session starts, the processor instructs display 140 to display instructions to relax (e.g., by text visually presented on a relaxing background and/or vocal instructions provided, e.g., on the background of tranquil music). The processor then identifies signals, received when the instructions to relax are displayed, as signals of the first kind. The processor may be further configured to replace the relaxation instructions to instructions to walk actively, e.g., after displaying the relaxation instructions for a predetermined time, after the patient walked passively a predetermined number of gait cycles, etc. The processor may identify signals, received when the instructions to walk actively are displayed, as signals of the second kind.

Identifying a first signal (or plurality of signals) as a signal (or signals) of the first kind and another signal (or plurality of signals) as a signal (or signals) of a second kind may be considered as distinguishing between signals of the first and second kind.

To carry out method 700, processor 150 or 330 may further be configured to

determine a net force as a difference between a force indicated by the first signals and a force indicated by the second signals. As discussed above, the net force may be determined for a plurality of gait cycle points. The net force may be determined, for example, by calculation according to the formula

Fₙₑₜ=F₂−F₁

In the above formula, Fₙₑₜ is the net force, F₂ is the force measured when the patient is instructed to move; and F₁ is the force measured when the patient is instructed to relax. The forces may be defined for each gait cycle point individually, or for some predefined cycle points individually, or for a group of gait cycle points (e.g., average forces over the points included in the plurality).

Finally, to carry out method 700, processor 150 or 330 may further be configured to act based on the net force determined. The action may include, for example, providing real-time feedback to the patient, instructing the robot how to move, and/or instructing the patient how to move, as discussed above in explaining method 700.

In some embodiments, an apparatus configured to carry out method 700 may further include a hoist, e.g., hoist 120. In such embodiments, the processor may be configured to control the hoist to lift the patient so as to reduce weight of the patient that rests on the patient's feet. The hoist may be activated by the processor automatically, for example, when an indication that a calibration starts, or by explicit instructions from a user (e.g., a therapist), provided, for example, through user interface 160. In some embodiments, the processor may stop lifting the patient when the entire weight of the patient is on the hoist. The processor may identify this point if, for example, the processor is configured to receive from the hoist data indicative of the weight carried by the hoist, and the processor is configured to identify when further lifting does not add to this weight.

In some embodiments, the forces of the first kind are measured when the entire weight of the patient is carried out by the hoist. The processor may be configured to identify the signals of the first kind as signals received when the entire weight of the patient is carried by the hoist.

Similarly, in some embodiments, the user may instruct the processor through the user interface to lower the hoist so that a portion of the weight of the patient is carried by the patient and/or to lift the patient by the hoist, e.g., so as to increase the portion of the patient's weight carried by the hoist. In some embodiments, the processor may be configured to determine what portion of the patient's weight is carried by the hoist at a particular moment, for example, by dividing the weight carried by the hoist at the particular moment by the full weight of the patient. The full weight of the patient may be measured as described above. In some embodiments, the full weight of the patient may be entered via user interface 160, e.g., based on weighing that took place before exercising on the gait rehabilitation apparatus started. The user interface may be configured to allow a user to instruct the processor to reduce (or enlarge) the height of the hoist so that a predetermined portion (e.g., 50%) of the patient's weight or a predetermined weight (e.g., 20 kg) is carried by the hoist (or by the patient). The processor may be configured to stop lowering (or heightening) the hoist when the predetermined portion of the patient's weight is carried by the hoist, based on calculation of the above ratio.

The processor may be configured to identify signals received from the sensor when a portion of the weight of the patient is carried by the patient as signals of the second kind.

Processor 150 or 330 may also be configured to take an action at a late point along a gait cycle based on net force determined at an early point along the gait cycle. For example, the processor may be configured to instruct the robot to slow down at the later point if the net force measured at the early point is below a threshold, and/or speed up at the late point if the net force determined at the early point is above a threshold.

### Examples of Generating and Executing Training Sessions

In some embodiments, a training session is generated by the apparatus based on input regarding the diagnosis and performance level of the patient. For example, the performance level may be determined based on performance in standardized tests, such as 10 meter walk test, Timed up and go test, and Berg balance test. A possible grouping of patients according to their achievements in one or more of these tests is provided in table 1 below:

TABLE 1

| | | | Group 3 | | | | | |
|---|---|---|---|---|---|---|---|---|
| | Group 4 | | MODERATE | | | | | |
| | SEVERE | | (minimum | | Group 2 | | | |
| | (wheelchair, 2 | | assistance, 1 | | MILD | | Group 1 | |
| | caregivers) | | caregiver | | Supervised | | INDEPENDENT | |
| 10 meter walk | 0.16-0.25 | m/s | 0.25-0.43 | m/s | 0.43-0.79 | m/s | 0.8-1.2 | m/s |
| Up and Go | >30 | sec | 20-30 | sec | 15-20 | sec | 0-14 | sec |
| Berg Balance | <18 | sec | 18-36 | sec | 36-45 | sec | 45-56 | sec |

In some embodiments, the processor generates for each patient a session program in accordance with the group to which the patient belongs. The group, or the achievements in the tests, may be entered by a therapist via an interface, e.g., interface 160 referred to in the context of FIG. 1A. The processor may select from a database a pre-planned session program based on the group. In some embodiments, the processor may modify the selected program to the individual patient, for example, based on achievements of the patient in preceding training session. The processor may generate an indication to the therapist, indicating the selected session program and its modifications, and the therapist may approve the suggestion or modify it.

In some embodiments, a session program may begin with a warm-up that includes guided walk, where the patient's legs are moved by the robot to walk on the treadmill, and the patient is required only to follow the robot. Program sessions of patients that belong to different groups may differ from each other, for example, by the speed of walking during this warm-up. For example, in some embodiments, for patients in group 4, the treadmill will go at 0.5 km/h; for group 3—at 0.8 km/h; for group 2—at lkm/h: and for group 1—at 1.2 km/h. The duration of the warm-up may also differ between the groups, for example, 5 minutes warm up for groups 3 and 4; and 2.5 minutes warm ups for groups 1 and 2. In some embodiments, if the sensors show that the patient performs very well during the first half of the warm-up (e.g., with compliance level above some predetermined threshold), the processor may produce a suggestion to the therapist to increase the speed of the walking, the weight bearing or, in some embodiments, the processor may do so without intervention of the therapist, optionally after indicating to the patient that his performance is excellent, and the speed is being raised. In some embodiments, if the sensors show that the patient has a high resistance or his symmetry in weight bearing is low during the first half of the warm-up (e.g., with compliance level below some predetermined threshold), the processor may produce a suggestion to the therapist to decrease the speed of the treadmill, decrease the patient weight bearing, or, in some embodiments, the processor may do so without intervention of the therapist. These and other features of the exemplary session program that may be generated based on severity of the patient's condition are detailed in the following tables.

TABLE 2
GROUP 4

| | Time | Speed | WB | |
|---|---|---|---|---|
| Mode | (minutes) | (km/h) | (%) | Gait Profile |
| GUIDED | 5 | 0.5 | 20 | Profile 1 |
| | 5 | 0.7 | 25 | Profile 2 |
| | 5 | 0.9 | 30 | Profile 3 |
| GUIDED + VR | 10 | 1 | 30 | Profile 4 |
| GUIDED | 5 | 0.5 | 20 | Profile 3 |

In the embodiment summarized in table 2, patients of group 4 (of severe condition) are trained only in the guided mode, and are not required to actively participate in moving their legs beyond what's needed to allow the robot to move them. The session may be made of several different parts, each characterized by a different speed, and with a different weight balance and with different gait profiles. A gait profile may include the range of motion angles through which the different joints (e.g., hips and knees) go through a gait cycle. The gait profile may determine the number of steps per minute at a given gait speed and/or be equivalent to a step size. Examples of gait profiles are provided below.

The weight balance indicates what percentage of the patient's body weight is carried by the patient, the rest being carried by the hoist. The changing of the body weight may, in some embodiments, be carried out manually. In some embodiments, it may be carried out automatically, under control of processor 150 (FIG. 1). In some embodiments, the processor controls display 140 to suggest the change in weight balance, and the therapist carries the suggested change, or any other change, or no change, in accordance with their best judgement. In the embodiment summarized in table 2, 10 minutes are planned for guided walking as described above, accompanied by training of the upper body, mainly the hands, in a mode named GUIDED+VR. The hands may be trained to reach virtual objects in a virtual reality setup. Virtual reality setups that may be used for training are described, for example, in Applicants' patent application titled VIRTUAL REALITY BASED REHABILITATION APPARATUSES AND METHODS, published as US patent application publication No. 2015-0133820, the entire contents of which are incorporated herein by reference. In the examples provided in the tables, the virtual reality is combined only with the GUIDED MODE, but in other examples it may be combined with any of the other modes, e.g., INITIATED, FOLLOW ASSIST, and FREE.

When a patient is trained in the GUIDED mode, the compliance level may be determined based on two factors: resistance forces exerted by the patient's legs against the robot, and the symmetry in weight bearing between the legs. The resistance forces may be reflected, for example, by the currents consumed by the motors moving the robot arms and/or by load cells at the hips and/or at the knees. In some embodiments, these forces are compared to normal values, forces exerted by healthy subjects, and the comparison results provide basis for determining the compliance ratio. The symmetry may be a ratio between the weights laid by the patient on each leg during the single limb support stage of gait. This may be reflected, for example, in results obtained from sensors in the sole, and/or in load cells at the hoist. In some embodiments, these two factors are equally weighted. In some other embodiments, these two factors are weighted so that one of them, for example, the symmetry, has a greater part in determining the compliance level than the other. The weight ratio between the two factors may be, for example, 40%:60%. In some embodiments, where virtual reality is used, the success rate in virtual reality tasks may also be taken into account in determining the compliance level. In some embodiments, the achievements in the virtual reality tasks is taken into consideration only when this is a main goal of the practice (e.g., with independent patients), and where the main goal is practicing the movement of the legs, the virtual reality is not considered in determining the compliance level, but used to keep the patient's interest and involvement in the training high.

### Examples of Gait Profiles

| | Hip flexion (°) | Hip extension(°) |
|---|---|---|
| Profile 1 | 11 | 11 |
| Profile 2 | 13 | 12 |
| Profile 3 | 15 | 14 |
| Profile 4 | 17 | 15 |
| Profile 5 | 19 | 16 |
| Profile 6 | 21 | 17 |

TABLE 3
GROUP 3

| | | | | Activity | |
|---|---|---|---|---|---|
| | Time | Speed | WB | Level | |
| Mode | (minutes) | (km/h) | (%) | (Kg) | Gait Profile |
| GUIDED | 2 | 0.7 | 20 | | Profile 2 |
| | 3 | 1 | 25 | | Profile 3 |
| | 2 | 1.2 | 30 | | Profile 4 |
| GUIDED + VR | 10 | 1 | 30 | | Profile 5 |
| FOLLOW ASSIST | 5 | 0.5-1 | 30 | 1-3 | Profile 6 |
| GUIDED | 5 | 0.7 | 30 | | Profile 5 |

In the embodiment summarized in table 3, patients of group 3 (of moderate condition) are trained in the guided mode, described above, and in the “follow assist” mode. In the “follow assist” mode, the patients are required not only to participate in moving their legs as needed to allow the robot to move them, but also to actively exert extra force, when the robot exerts less. For example, the patient may be alerted that the robot is going to exert less force, and about 1 second after the warning, the robot decreases the force it exerts, so the patient has to increase the force exerted by himself in order to retain the gait speed. If the patient succeeds in increasing the force they exert during a res-set time window from the alert, the robot continues walking at somewhat higher speed, to provide the patient with sensory feedback on his success. Working in this mode may challenge the cardiovascular system of the patient. In some embodiments, heart rate of the patient is monitored, and the processor alerts the therapist when the heart rate approaches a predetermined value, e.g., of 0.6×(220-age in years). Work in the follow assist mode is characterized by an activity level, designating the level of participation expected from the patient. The speed in this mode is given in range format, since as the patient succeeds, the speed goes up. When the patient fails, the robot and treadmill may stop for a while, and continue again from the speed they had before the failure, or from somewhat slower speed. The success rate in assisting the robot in gait training may also be taken into account in determining the compliance level in this working mode. For example, the compliance level may be provided by a grade made of 50% follow assist success rate, 30% symmetry, and 20% deviation of resistance forces from what's usual with healthy subjects.

TABLE 4
GROUP 2

| | | | | | Activity |
|---|---|---|---|---|---|
| | Time | Speed | WB | | Level |
| Mode | (minutes) | (km/h) | (%) | Gait Profile | (Kg) |
| GUIDED | 2.5 | 1 | 20 | Profile 2 | |
| INITIATED | 5 | 1 | 30 | Profile 3 | 1-3 |
| LR-MS | | | | | |
| Every 2 cycles | | | | | |
| INITIATED | 5 | 1 | 30 | Profile 3 | 1-3 |
| MS-TS | | | | | |
| INITIATED | 5 | 1 | 30 | Profile 3 | 1-3 |
| PS-IS | | | | | |
| FOLLOW | 5 | 0.5-1.2 | 30 | Profile 4 | 1-3 |
| ASSIST | | | | | |
| GUIDED + VR | 5 | 1 | 30 | Profile 5 | |
| FREE | 10 | 1 | 30 | | |

In the embodiment summarized in table 4, patients of group 2 (of mild condition) are trained in two modes additionally to the GUIDED mode and “follow assist” mode described above. These two modes are the INITIATED mode and the FREE mode.

In initiated mode, the patient is instructed to walk actively at a certain part of the gait cycle, and receives feedback on their success, e.g., via interface 160. The gait cycle parts mentioned in the table are: LR-MS (loading response—mid-stance); MS-TS (mid-stance—terminal stance); and PS-IS (pre-swing—initial swing). Each of these parts of the gait cycle (which may also be considered gait events), is trained for 5 minutes. The training may take place, for example, every second gait cycle. The compliance level may be determined taking into consideration the success rate in the INITIATED tasks, of actively replacing some of the force that in GUIDED mode is provided by the robot alone.

In the FREE mode, the patient is freed from the robot, and walks on the treadmill only with the help of the hoist, that carries a portion of the patient's weight. The speed is determined by the treadmill alone. In addition, one may determine a target step size. In some embodiments, the target step size is determined manually by the therapist. In some embodiments, the processor suggests to the therapist a target step size, for example, based on the target step size of the gait profile trained last, and the compliance level at that gait profile. The target step size may be indicated to the patient using the virtual reality environment, which may also provide feedback whether the target is achieved or by how much it is missed, for each foot. The compliance level may be determined based on the symmetry between the legs (as reflected, for example, in readings of the sensors at the sole and/or in video captured by cameras positioned, for example, at the base of the treadmill. The video may be image-processed to extract from it data on symmetry of the gait, step size, etc. Compliance level in the free mode may be determined based on the symmetry in the gait and the step size in comparison to the target step size. The symmetry may be in weight bearing and step length. Data for determining the compliance level may be received, for example, from sensors in the sole and/or cameras.

During each part of the training, the processor may suggest the therapist to change the weight balance, the speed, and/or the gait profile based on the compliance level achieved so far in that part of the training. For example, during the training in FREE mode, if the compliance level is above some predetermined threshold, the speed may be increased. In another example, in the INITIATED mode, if the compliance level is above some predetermined threshold, the weight balance, the activity level, and/or the gait profile may be increased.

Finally, table 5 provides an example of a training session of patients in group 1 (independent patients) according to some embodiments of the invention.

TABLE 5
GROUP 1

| | | | | Activity | |
|---|---|---|---|---|---|
| | Time | Speed | WB | Level | |
| Mode | (minutes) | (km/h) | (%) | (Kg) | Gait Profile |
| GUIDED | 2.5 | 1.2 | 20 | | Profile 3 |
| INITIATED | 5 | 1 | 30 | 1-3 | Profile 4 |
| LR-MS | | | | | |
| INITIATED | 5 | 1 | 30 | 1-3 | Profile 4 |
| MS-TS | | | | | |
| INITIATED | 5 | 1 | 30 | 1-3 | Profile 4 |
| PS-IS | | | | | |
| FOLLOW | 5 | 0.5-1.5 | 30 | 1-3 | Profile 5 |
| ASSIST | | | | | |
| GUIDED + VR | 5 | 1.2 | 30 | | Profile 6 |
| FREE | 15 | 1 | 30 | | |

Tables 2 to 5 illustrate training sessions planned, according to some embodiments, based on the severity of the patient's condition. In some embodiments, diagnosis may also be considered in planning the session. For example, the follow assist mode and the initiated mode may train one or both legs. In patients with unilateral injuries (e.g., after stroke, total knee replacement, total hip replacement, etc.) the session program may include training of the injured leg only, or mainly. In some embodiments, healthy legs may be trained to allow the patient to feel what he is asked to do. Also, in patients with unilateral injuries, the symmetry between the legs may be or particular importance, and may have a larger weight in determining the compliance level than in bilateral injuries where both parts are similarly injured.

In some embodiments of the invention, tables similar to tables 2-5 may be held in a database accessible to processor 150. The database may include tables for patients of different severity, different diagnosis, different ages, genders, etc. In some embodiments, processor 150 may be configured to generate a table based on achievements of the patient in preceding training sessions, for example, by modifying an existing table. For example, the processor may suggest to the therapist initial speeds, weight balances, gait profile and activity levels based on the same parameters trained in a preceding session, and the compliance levels achieved during that training. In some embodiments, the processor may suggest to the therapist to change the severity level of a patient, based on the patient's performance in training sessions planned for his severity level. For example, the processor may suggest, e.g., via interface 160, that a patient will be advance from group 4 to group 3, etc. The therapist may decide to train the patient with session program designed for the more advanced group with or without taking one or more of the tests referred to in table 1.

### Examples of Displaying Forces Exerted by Patient's Legs

As noted above, in some embodiments, the forces exerted by the patient against the robot may be taken into account in determining the compliance level of the patient in all training methods that involve the robot. In some embodiments, these forces may be displayed to the therapist to allow them better understanding of the muscle resistance that is developed during the gait training, and for modifying the training session parameters by the therapist to the individual patient in order to reduce the resistance. In some examples, the therapist may change gait profile for the patient based on the forces the patient exerts.

In some embodiments, each joint is moved by its own motor, and each motor provides the processor with data indicative of the electrical currents consumed by the motor in real time. These currents may be displayed to the therapist as four different displays (one for each knee and one for each hip), so the therapist can see if there is a particular resistance around one of the joints at a specific timing of the gait. In some embodiments, the forces may be compared to forces measured to be exerted by healthy subjects, so if there is a natural tendency to exert more force at some portion of the gait cycle, this tendency will not affect the results displayed. In some embodiments, the currents are shown along the gait cycle, for example, the gait cycle is divided to 100 portions, and a graph with 100 points is shown, where each point presents a current value at a corresponding portion of the gait cycle, and the display is refreshed each gait cycle.

In some embodiments, deviations of the currents or forces from those measured during gait of healthy subjects are shown in different colors. For example, green may indicate forces of the range expected from healthy subjects, yellow may indicate somewhat larger forces, and red may indicate considerably larger forces. This way, the therapist may easily distinguish at which portions of a gait cycle the patient faces more muscle resistance, and around which joint.

In some embodiments, the data are presented by an image of a human leg, and data indicative of forces exerted by a motor that moves a part of a leg of the patient is presented by coloring the respective part of the leg in the image, so that different colors represent different ranges of forces. In dome embodiments, the data are presented when the human leg moves as in the gait cycle, which may help the therapist even more to understand which portion of the gait cycle is most problematic to the patient, and at what joint.

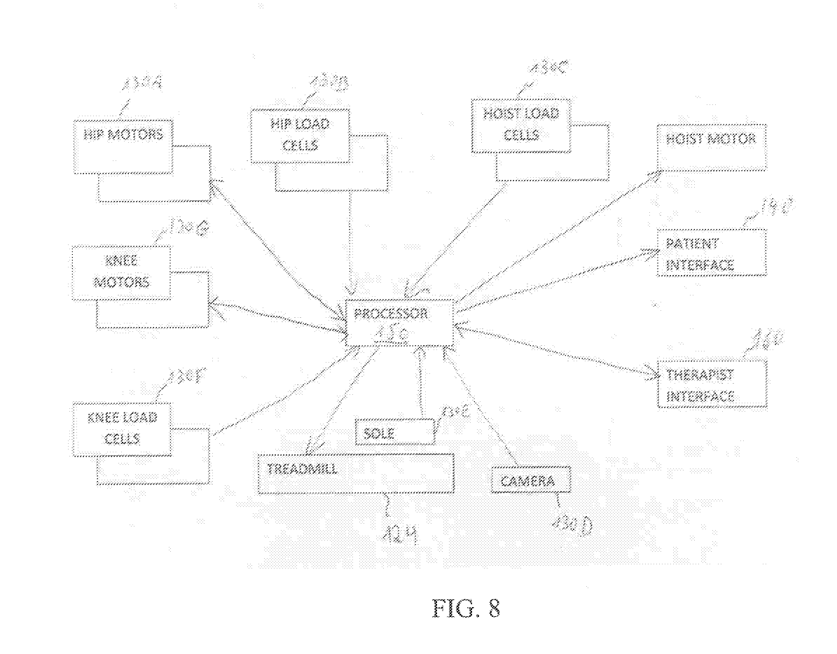

FIG. 8 is a block diagram describing a training apparatus according to some embodiments of the invention. As may be see, central to the functioning of the apparatus is at least one processor 150. While only one processor is illustrated in the figure, but as mentioned before, any processor recited herein may be replaced with a plurality of processors that together are configured to carry out its functions. Processor 150 receives inputs from sensors 130A-130G. These include amperemeters at the hip motors and knee motors (e.g., one motor for each hip and one motor for each knee); load cells at the hips and at the knees, load cells at the hoist, sensor at the sole of the patient's shoe, and a camera. The processor processes data received from sensors 130A to 130G, to generate suggestions for actions to user interface 160, serving the therapist. The processor may receive instructions from the therapist via interface 160, and based on these instructions, control the moving parts of the apparatus to execute a session program. The moving parts include the hip and knee motors (and respective robotic arms and cuffs, shown in FIG. 1B), and treadmill 124. Processor 150 may include a memory, or may be connected to a memory (e.g., via an Internet connection) storing a database of session programs (e.g., of the kind summarized in tables 2-5 above), and rules for calculating compliance levels and for suggesting, best on calculated compliance levels, actions such as speed increase or decrease; increase or decrease of weight balance, or changing a gaiting profile. Processor 150 may also be configured to display data received from the sensors, optionally in processed form to therapist interface 160. This apparatus allows for automatic or semi-automatic generation and execution of training sessions. In this case, semi-automatic refers to automatically suggesting training sessions and actions during their execution, and carrying out the suggestions only after being confirmed by the therapist. Whenever in the present disclosure it is mentioned that something is executed automatically, it covers also semi-automatic execution, and any instruction from the processor to any of the moving parts may require receiving first the authorization of the therapist.

In the foregoing Description of Exemplary Embodiments, various features are grouped together in a single embodiment for purposes of streamlining the disclosure. This method of disclosure is not to be interpreted as reflecting an intention that the claimed invention requires more features than are expressly recited in each claim. Rather, inventive aspects may lie in less than all features of a single foregoing disclosed embodiment. Moreover, it will be apparent to those skilled in the art from consideration of the specification and practice of the present disclosure that various modifications and variations can be made to the disclosed apparatuses and methods without departing from the scope of the invention, as claimed. For example, one or more steps of a method and/or one or more components of an apparatus or a device may be omitted, changed, or substituted without departing from the scope of the invention. Thus, it is intended that the specification and examples be used as examples only, with a true scope of the present disclosure being indicated by the following claims and their equivalents.

It will be appreciated that the above described methods may be varied in many ways, including, omitting or adding steps, changing the order of steps and the types of devices used. In addition, a multiplicity of various features, both of method and of devices have been described. In some embodiments mainly methods are described, however, apparatuses adapted for performing the methods are also considered to be within the scope of the invention.

It should be appreciated that different features may be combined in different ways. In particular, not all the features shown above in a particular embodiment are necessary in every similar embodiment of the invention. Further, combinations of the above features are also considered to be within the scope of some embodiments of the invention. Also, within the scope is hardware, software and computer readable-media including such software which is used for carrying out and/or guiding the steps described herein, such as control of patient's leg movement, instructing the patient to act, and providing feedback.

Section headings are provided for assistance in navigation and should not be considered as necessarily limiting the contents of the section. When used in the following claims, the terms “comprises”, “includes”, “have” and their conjugates mean “including but not limited to”. It should also be rioted that the device is suitable for both males and female, with male pronouns being used for convenience.

It will be appreciated by a person skilled in the art that the present invention is not limited by what has thus far been described. Rather, the scope of the present invention is limited only by the following claims.

1. A computer-implemented method for training a patient in moving, the method comprising:
obtaining a session program for the patient, the session program comprising a plurality of exercises and the order by which they are to be practiced by the patient;
receiving results of measurements made during an early stage of training according to the session program, said measurements being indicative of parameters characterizing the moving of the patient; and
executing a later stage of the session program based on the results received during the early stage of the training.

2. The computer-implemented method of claim 1, wherein the session program includes a first exercise; a second exercise; and instructions to execute the first exercise before executing the second exercise, and the method comprising:
executing the first exercise;
during execution of the first exercise, receiving results of measurements indicative of a compliance level of the patient in practicing the first exercise;
and switching to executing the second exercise after the results received indicate a compliance level equal to or higher than a target compliance level.

3. The computer-implemented method of claim 1, wherein the session program includes a first exercise; a second exercise; and instructions to execute the first exercise before executing the second exercise, and the method comprising:
executing the second exercise after executing the first exercise;
during execution of the second exercise, receiving results of measurements indicative of a compliance level of the patient in practicing the second exercise;
and switching to executing the first exercise again, after the results received indicate a compliance level lower than a target compliance level.

4. The computer-implemented method of claim 1, wherein obtaining the session program comprises:
receiving input indicative of at least one of diagnosis of the patient and performance level of the patient; and
generating the session program based on the input received.

5. The computer-implemented method of claim 1, wherein the session program includes, for each of the plurality of exercises, at least one target compliance level.

6. The computer-implemented method of claim 5, wherein receiving results of measurements comprises receiving from sensors configured to sense forces exerted by the patient during the training.

7. The computer-implemented method of claim 1, wherein the session program includes a plurality of minimal durations, each of the plurality of minimal durations is associated with a corresponding one or more exercises of the plurality of exercises, and the method comprises:
estimating a compliance level of the patient based on results received during execution of an exercise after the exercise is executed for the minimal duration associated with said exercise.

8. An apparatus for training a patient in moving by executing a session program comprising a plurality of exercises and the order by which the exercises are to be practiced by the patient, the apparatus comprising a processor configured to:
receive results of measurements made during an early stage of training according to the session program, said measurements being indicative of parameters characterizing the moving of the patient; and
execute a later stage of the session program based on the results received during the early stage of the training.

9. An apparatus according to claim 8, wherein the session program includes a first exercise; a second exercise; and instructions to execute the first exercise before executing the second exercise, and the processor is configured to:
provide the patient instructions to practice the first exercise;
during execution of the first exercise by the patient, receive results of measurements indicative of a compliance level of the patient; and
providing the patient instructions to practice the second exercise after the results received indicate a compliance level equal to or higher than a target compliance level.

10. The apparatus of claim 8, wherein the session program includes a first exercise; a second exercise; and instructions to execute the first exercise before executing the second exercise, and the processor is configured to:
provide the patient instructions to practice the second exercise after practicing the first exercise;
during practicing of the second exercise by the patient, receive results of measurements indicative of a compliance level of the patient; and
provide the patient instructions to execute the first exercise again, after the results received indicate a compliance level lower than a target compliance level.

11. The apparatus of claim 8, wherein the processor is configured to obtain the session program by generating the session program based on input indicative of at least one of diagnosis of the patient and performance level of the patient.

12. The apparatus of claim 8, wherein the session program includes, for each of the plurality of exercises, at least one target compliance level.

13. The apparatus of claim 12, which comprises sensors configured to sense forces exerted by the patient during the training, and the processor is configured to receive the results of measurements from the sensors.

14. The apparatus of claim 8, wherein the session program includes a plurality of minimal durations, each of the plurality of minimal durations is associated with a corresponding one of the plurality of exercises, and the processor is configured to:
estimate a compliance level of the patient based on results received during execution of an exercise after the exercise is executed for the minimal duration associated with said exercise.
